(19) United States
(12) Patent Application Publication
(10) Pub. No.: US 2025/0176920 A1
(43) Pub. Date: Jun. 5, 2025

(54) CHRONIC PULMONARY DISEASE PREDICTION FROM AUDIO INPUT BASED ON INHALE-EXHALE PAUSE SAMPLES USING ARTIFICIAL INTELLIGENCE

(71) Applicant: SONY GROUP CORPORATION, TOKYO (JP)

(72) Inventors: SACHIN KUMAR AGRAWAL, BENGALURU (IN); AYUSH KUMAR GHADIYA, BENGALURU (IN); MEGHA KETANKUMAR GORIYA, BENGALURU (IN)

(21) Appl. No.: 18/529,903

(22) Filed: Dec. 5, 2023

(51) Int. Cl. A61B 5/00; A61B 5/087; A61B 7/00; G06T 11/20; G10L 21/0232; G10L 25/06; G10L 25/21; G10L 25/30; G10L 25/66

(52) U.S. Cl. CPC A61B 5/7275; A61B 5/087; A61B 5/7257; A61B 5/7264; A61B 7/003; G06T 11/206; G10L 21/0232; G10L 25/06; G10L 25/21; G10L 25/30; G10L 25/66

(57) ABSTRACT

An electronic device and method for chronic pulmonary disease prediction from audio input based on short-winded breath determination using artificial intelligence is disclosed. The electronic device receives an audio input associated with a user. The electronic device applies an Artificial Intelligence (AI) model to determine a first set of inhale-exhale pause samples. The electronic device selects an inhale-exhale pause sample from the first set of inhale-exhale pause samples. The electronic device applies a generative adversarial network (GAN) model on the selected inhale-exhale pause sample. The electronic device generates a flow volume curve associated with the selected inhale-exhale pause sample based on the application of the GAN model. The electronic device determines one or more voice spirometer parameters based on the generated flow volume curve. The electronic device renders the determined one or more voice spirometer parameters on a display device associated with the electronic device.

### FIELD

Various embodiments of the disclosure relate to chronic pulmonary disease prediction. More specifically, various embodiments of the disclosure relate to an electronic device and method for chronic pulmonary disease prediction from audio input based on inhale-exhale pause samples using artificial intelligence.

### BACKGROUND

Industrial growth and increased vehicular traffic have led to an increase in respiratory diseases, such as, pulmonary fibrosis, asthma, and chronic obstructive pulmonary disease (COPD). Such diseases may be mainly caused due to air pollution, smoking, agricultural pesticides, and industrial chemicals. Typically, a healthcare professional may recommend spirometry test to a patient. Spirometry is a commonly used test to measure an amount of air that enters and leaves the lungs, before and after use of an inhaled bronchodilator. However, spirometry may not provide etiological diagnosis, may fail to detect obstructive-restrictive defect, and may depend on the patient's effort, and require skilled operators. Further, spirometry may be costly and may cause annoyance for the patients as the diagnosis may require a multiple breathing maneuvers.

Limitations and disadvantages of conventional and traditional approaches will become apparent to one of skill in the art, through comparison of described systems with some aspects of the present disclosure, as set forth in the remainder of the present application and with reference to the drawings.

### SUMMARY

An electronic device and method for chronic pulmonary disease prediction from audio input based on inhale-exhale pause samples using artificial intelligence, and/or described in connection with, at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a block diagram that illustrates an exemplary network environment for chronic pulmonary disease prediction from audio input based on inhale-exhale pause samples using artificial intelligence, in accordance with an embodiment of the disclosure.

FIG. 2 is a block diagram that illustrates an exemplary electronic device of FIG. 1, in accordance with an embodiment of the disclosure.

FIGS. 3A and 3B diagrams that collectively illustrate an exemplary processing pipeline for chronic pulmonary disease prediction from audio input based on inhale-exhale pause samples using artificial intelligence, in accordance with an embodiment of the disclosure.

FIGS. 4A and 4B are diagrams that together illustrate an exemplary scenario for chronic pulmonary disease prediction from audio input based on inhale-exhale pause samples using artificial intelligence, in accordance with an embodiment of the disclosure.

FIG. 5 is a flowchart that illustrates operations of an exemplary method for denoising audio input, in accordance with an embodiment of the disclosure.

FIG. 6A is a diagram that illustrates an exemplary scenario for determination of a first set of inhale-exhale pause samples, in accordance with an embodiment of the disclosure.

FIG. 6B is a diagram that illustrates an exemplary scenario of a bar graph representation for the first set of inhale-exhale pause samples of FIG. 6A, in accordance with an embodiment of the disclosure.

FIG. 7A is a diagram that illustrates an exemplary scenario for a determination of an intermediate set of inhale-exhale pause samples based on an application of an artificial intelligence (AI) model, in accordance with an embodiment of the disclosure.

FIG. 7B is a diagram that illustrates an exemplary scenario for a selection of an inhale-exhale pause sample from a first set of inhale-exhale pause samples based on determination of energies, in accordance with an embodiment of the disclosure.

FIG. 8 is a diagram that illustrates an exemplary scenario for a selection of an inhale-exhale pause sample from a first set of inhale-exhale pause samples based on energy-based rankings, in accordance with an embodiment of the disclosure.

FIG. 9 is a diagram that illustrates an exemplary processing pipeline for generation of a flow volume curve, in accordance with an embodiment of the disclosure.

FIG. 10 is a diagram that illustrate exemplary scenario for determination of an adaptive cutoff-frequency, in accordance with an embodiment of the disclosure.

FIGS. 11A and 11B are diagrams that illustrate exemplary scenarios of flow volume curves, in accordance with an embodiment of the disclosure.

FIGS. 12A and 12B are diagrams that collectively illustrate an exemplary scenario for generation of an optimized signal, in accordance with an embodiment of the disclosure.

FIG. 13 is a diagram that illustrate exemplary scenario for determination of a breathing condition and a chronic disease condition, in accordance with an embodiment of the disclosure.

FIG. 14 is a diagram that illustrate exemplary scenario for determination of a chronic disease condition, in accordance with an embodiment of the disclosure.

FIG. 15 is a diagram that illustrate exemplary scenario for determination of a vocal disorder, in accordance with an embodiment of the disclosure.

FIG. 16 is a flowchart that illustrates operations of an exemplary method for chronic pulmonary disease prediction from audio input based on inhale-exhale pause samples using artificial intelligence, in accordance with an embodiment of the disclosure.

### DETAILED DESCRIPTION

The following described implementation may be found in an electronic device and a method for chronic pulmonary disease prediction from audio input based on inhale-exhale pause samples using artificial intelligence. Exemplary aspects of the disclosure may provide an electronic device (for example, a server, a desktop, a laptop, or a personal computer) that may receive an audio input associated with a user. The electronic device may apply an Artificial Intelligence (AI) model on the received audio input. The electronic device may determine a first set of inhale-exhale pause samples based on the application of the AI model, wherein each inhale-exhale pause sample of the determined first set of inhale-exhale pause samples may correspond to a time interval between consecutive inhale and exhale breathlessness samples. The electronic device may select an inhale-exhale pause sample from the first set of inhale-exhale pause samples. The electronic device may apply a generative adversarial network (GAN) model on the selected inhale-exhale pause sample. The electronic device may generate a flow volume curve associated with the selected inhale-exhale pause sample based on the application of the GAN model. The electronic device may determine one or more voice spirometer parameters based on the generated flow volume curve. The electronic device may render the determined one or more voice spirometer parameters on a display device associated with the electronic device.

Typically, a diagnosis of the COPD is done by spirometry. It may be appreciated that the spirometry may diagnose the COPD based on a measurement of an amount of air that enters and leaves the lungs, before and after a use of an inhaled bronchodilator. However, spirometry does not provide etiological diagnosis, fails to detect obstructive-restrictive defect, provides diagnosis based on a patient's effort, and requires skilled operators. Moreover, spirometry may be costly and may cause annoyance to the patients as diagnosis with spirometry may require a multiple number of breathing maneuvers.

In order to non-invasively diagnose the COPD, the present disclosure introduces a method of chronic pulmonary disease prediction from audio input based on inhale-exhale pause samples using artificial intelligence. The electronic device of the present disclosure may determine the first set of inhale-exhale pause samples based on the application of the AI model to the received audio input. Thereafter, the electronic device may select the inhale-exhale pause sample from the first set of inhale-exhale pause samples. The application of the AI model may reduce a processing-time needed for selection of the inhale-exhale pause sample. The selected inhale-exhale pause sample may be applied to the GAN model to generate the flow volume curve. The flow volume curve may be used to determine the one or more voice spirometer parameters.

In some embodiments, the generated flow volume curve and the determined one or more voice spirometer parameters may be used to determine a breathing condition and a chronic disease condition. Further, in some embodiments, the electronic device may determine a vocal disorder. Therefore, the disclosed electronic device may be used effortlessly by patients as well as doctors or nurses for determination of the one or more voice spirometer parameters. Also, as the AI model may automatically select the inhale-exhale pause sample, time needed in manual selection of the inhale-exhale pause sample may be reduced. Moreover, the disclosed electronic device may generate an accurate flow volume curve that may be used for accurate interpretation of the one or more voice spirometer parameters. Furthermore, the patients may use the disclosed electronic device for self-diagnosis of the chronic disease condition. Thus, the disclosed electronic device may need minimal medical-expert intervention during the diagnosis of the chronic disease condition or the vocal disorder. The disclosed electronic device may enable an early diagnosis of the chronic disease condition in a time-efficient and cost-efficient manner.

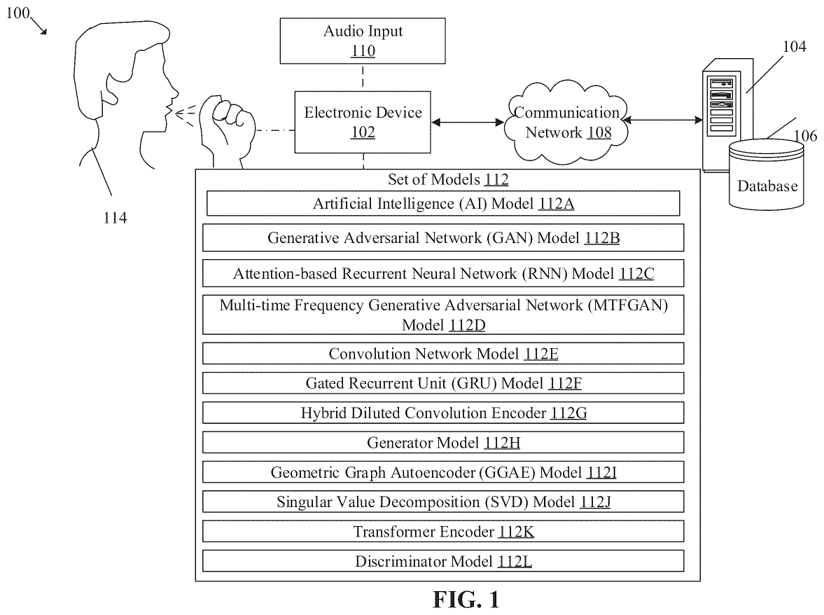

FIG. 1 is a block diagram that illustrates an exemplary network environment for chronic pulmonary disease prediction from audio input based on inhale-exhale pause samples using artificial intelligence, in accordance with an embodiment of the disclosure. With reference to FIG. 1, there is shown a network environment 100. The network environment 100 may include an electronic device 102, a server 104, a database 106, and a communication network 108. In FIG. 1, there is further shown audio input 110. The electronic device 102 may be associated with a set of models 112. The set of models 112 may include an Artificial Intelligence (AI) model 112A, a generative adversarial network (GAN) model 112B, an attention-based recurrent neural network (RNN) model 112C, a multi-time frequency generative adversarial network (MTFGAN) model 112D, a convolution network model 112E, a gated recurrent unit (GRU) model 112F, a hybrid diluted convolution encoder 112G, a generator model 112H, a geometric graph autoencoder (GGAE) model 1121, a singular value decomposition (SVD) model 112J, and a transformer encoder 112K. The electronic device 102 and the server 104 may be communicatively coupled to each another, via the communication network 108. In FIG. 1, there is further shown a user 114 who may be associated with and/or may operate the electronic device 102.

The electronic device 102 may include suitable logic, circuitry, interfaces, and/or code that may be configured to receive an audio input associated with a user, such as, the user 114. The electronic device 102 may apply the Artificial Intelligence (AI) model 112A on the received audio input 110. The electronic device 102 may determine a first set of inhale-exhale pause samples based on the application of the AI model 112A, wherein each inhale-exhale pause sample of the determined first set of inhale-exhale pause samples may correspond to a time interval between consecutive inhale and exhale breathlessness samples. The electronic device 102 may select an inhale-exhale pause sample from the first set of inhale-exhale pause samples. The electronic device 102 may apply the GAN model 112B on the selected inhale-exhale pause sample. The electronic device 102 may generate a flow volume curve associated with the selected inhale-exhale pause sample based on the application of the GAN model 112B. The electronic device 102 may determine one or more voice spirometer parameters based on the generated flow volume curve. The electronic device 102 may render the determined one or more voice spirometer parameters on a display device associated with the electronic device 102.

Examples of the electronic device 102 may include, but are not limited to, a computing device, a smartphone, a cellular phone, a mobile phone, a gaming device, a mainframe machine, a server, a computer workstation, and/or a consumer electronic (CE) device.

The server 104 may include suitable logic, circuitry, and interfaces, and/or code that may be configured to apply the AI model 112A on the received audio input 110. The server 104 may determine the first set of inhale-exhale pause samples based on the application of the AI model 112A. The server 104 may select the inhale-exhale pause sample from the determined first set of inhale-exhale pause samples. The server 104 may apply the GAN model 112B on the selected inhale-exhale pause sample. The server 104 may generate the flow volume curve associated with the selected inhale-exhale pause sample based on the application of the GAN model 112B. The server 104 may determine one or more voice spirometer parameters based on the generated flow volume curve. The server 104 may render the determined one or more voice spirometer parameters on the display device associated with the electronic device 102.

In one or more embodiments, the server 104 may store the set of models 112, the audio input 110, and/or the determined first set of inhale-exhale pause samples. Further, the server 104 may execute at least one operation associated with the electronic device 102. The server 104 may be implemented as a cloud server and may execute operations through web applications, cloud applications, HTTP requests, repository operations, file transfer, and the like. Other example implementations of the server 104 may include, but are not limited to, a database server, a file server, a web server, a media server, an application server, a mainframe server, or a cloud computing server.

In at least one embodiment, the server 104 may be implemented as a plurality of distributed cloud-based resources by use of several technologies that are well known to those ordinarily skilled in the art. A person with ordinary skill in the art will understand that the scope of the disclosure may not be limited to the implementation of the server 104 and the electronic device 102, as two separate entities. In certain embodiments, the functionalities of the server 104 can be incorporated in its entirety or at least partially in the electronic device 102 without a departure from the scope of the disclosure. In certain embodiments, the server 104 may host the database 106. Alternatively, the server 104 may be separate from the database 106 and may be communicatively coupled to the database 106.

The database 106 may include suitable logic, interfaces, and/or code that may be configured to store the determined one or more voice spirometer parameters. In certain scenarios, the database 106 may also store the set of models 112. The database 106 may be stored or cached on a device, such as a server (e.g., the server 104) or the electronic device 102. The device storing the database 106 may be configured to receive (e.g., from the electronic device 102 and/or the server 104) a query for the determined one or more voice spirometer parameters (and/or the set of models 112). In response, the device that stores the database 106 may retrieve and provide the determined one or more voice spirometer parameters (and/or the set of models 112) to the electronic device 102 and/or the server 104.

In some embodiments, the database 106 may be hosted on a plurality of servers stored at same or different locations. The operations of the database 106 may be executed using hardware, including a processor, a microprocessor (e.g., to perform or control performance of one or more operations), a field-programmable gate array (FPGA), or an application-specific integrated circuit (ASIC). In some other instances, the database 106 may be implemented using software.

The communication network 108 may include a communication medium through which the electronic device 102 and the server 104 may communicate with each another. The communication network 108 may be one of a wired connection or a wireless connection. Examples of the communication network 108 may include, but are not limited to, the Internet, a cloud network, Cellular or Wireless Mobile Network (such as Long-Term Evolution and 5^{th} Generation (5G) New Radio (NR)), a satellite network (such as, a network of a set of Low Earth Orbit satellites), a Wireless Fidelity (Wi-Fi) network, a Personal Area Network (PAN), a Local Area Network (LAN), or a Metropolitan Area Network (MAN). Various devices in the network environment 100 may be configured to connect to the communication network 108 in accordance with various wired and wireless communication protocols. Examples of such wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity (Li-Fi), 802.16, IEEE 802.11s, IEEE 802.11g, multi-hop communication, wireless access point (AP), device to device communication, cellular communication protocols, and Bluetooth (BT) communication protocols.

The AI model 112A may include suitable logic, interfaces, and/or code that may be applied on the on the received audio input 110. The AI model 112A may determine the first set of inhale-exhale pause samples. In an embodiment, the AI model 112A may be a machine learning (ML) model such as a classifier model which may be trained to identify a relationship between inputs, such as features in a training dataset and output labels, such as the first set of inhale-exhale pause samples. The AI model 112A may be defined by its hyper-parameters, for example, number of weights, cost function, input size, number of layers, and the like. The parameters of the AI model 112A may be tuned and weights may be updated so as to move towards a global minima of a cost function for the AI model 112A. After several epochs of the training on the feature information in the training dataset, the AI model 112A may be trained to output a prediction/classification result for a set of inputs. The prediction result may be indicative of a class label for each input of the set of inputs (e.g., input features extracted from new/unseen instances).

The AI model 112A may include electronic data, which may be implemented as, for example, a software component of an application executable on the electronic device 102. The AI model 112A may rely on libraries, external scripts, or other logic/instructions for execution by a processing device, such as the electronic device 102. The AI model 112A may include code and routines configured to enable a computing device, such as the electronic device 102 to perform one or more operations, such as determination of the first set of inhale-exhale pause samples. Additionally or alternatively, the AI model 112A may be implemented using hardware including a processor, a microprocessor (e.g., to perform or control performance of one or more operations), a field-programmable gate array (FPGA), or an application-specific integrated circuit (ASIC). Alternatively, in some embodiments, the AI model 112A may be implemented using a combination of hardware and software.

In an embodiment, the AI model 112A may be a neural network. The neural network may be a computational network or a system of artificial neurons, arranged in a plurality of layers, as nodes. The plurality of layers of the neural network may include an input layer, one or more hidden layers, and an output layer. Each layer of the plurality of layers may include one or more nodes (or artificial neurons, represented by circles, for example). Outputs of all nodes in the input layer may be coupled to at least one node of hidden layer(s). Similarly, inputs of each hidden layer may be coupled to outputs of at least one node in other layers of the neural network. Outputs of each hidden layer may be coupled to inputs of at least one node in other layers of the neural network. Node(s) in the final layer may receive inputs from at least one hidden layer to output a result. The number of layers and the number of nodes in each layer may be determined from hyper-parameters of the neural network. Such hyper-parameters may be set before, while training, or after training the neural network on a training dataset.

Each node of the neural network may correspond to a mathematical function (e.g., a sigmoid function or a rectified linear unit) with a set of parameters, tunable during training of the network. The set of parameters may include, for example, a weight parameter, a regularization parameter, and the like. Each node may use the mathematical function to compute an output based on one or more inputs from nodes in other layer(s) (e.g., previous layer(s)) of the neural network. All or some of the nodes of the neural network may correspond to same or a different same mathematical function.

In training of the neural network, one or more parameters of each node of the neural network may be updated based on whether an output of the final layer for a given input (from the training dataset) matches a correct result based on a loss function for the neural network. The above process may be repeated for same or a different input till a minima of loss function may be achieved, and a training error may be minimized. Several methods for training are known in art, for example, gradient descent, stochastic gradient descent, batch gradient descent, gradient boost, meta-heuristics, and the like.

The GAN model 112B may be a machine learning (ML) model that may be applied on the selected inhale-exhale pause sample for generation of the flow volume curve associated with the selected inhale-exhale pause sample. In another embodiment, the GAN model 112B may be a neural network. Details related to the ML model and the neural network associated with the GAN model 112B are similar to the details of the ML model and the neural network of the AI model 112A. Hence, the details related to the ML model and the neural network of the attention-based RNN model 112C are skipped for the sake of brevity of the disclosure.

The attention-based RNN model 112C may be a ML model that may be applied on the selected inhale-exhale pause sample for determination of an adaptive cutoff-frequency. In another embodiment, the attention-based RNN model 112C may be a neural network. Details related to the ML model and the neural network associated with the attention-based RNN model 112C are similar to the details of the ML model and the neural network of the AI model 112A. Hence, the details related to the ML model and the neural network of the attention-based RNN model 112C are skipped for the sake of brevity of the disclosure.

The MTFGAN model 112D may be a ML model that may be applied on determined multiple time-frequency spectrums for generation of an optimized signal. In another embodiment, the MTFGAN model 112D may be a neural network. Details related to the ML model and the neural network associated with the MTFGAN model 112D are similar to the details of the ML model and the neural network of the AI model 112A. Hence, the details related to the ML model and the neural network of the MTFGAN model 112D are skipped for the sake of brevity of the disclosure.

The convolution network model 112E may be applied on temporal set of features for determination of a set of feature embeddings. In an embodiment, the convolution network model 112E may be a neural network. Details related to the neural network associated with the convolution network model 112E are similar to the details of the neural network of the AI model 112A. Hence, the details related to the neural network of the convolution network model 112E are skipped for the sake of brevity of the disclosure.

The GRU model 112F may be applied on a set of attention features for determination of a set of frequency spectrums. In an embodiment, the GRU model 112F may be a neural network. Details related to the neural network associated with the GRU model 112F are similar to the details of the neural network of the AI model 112A. Hence, the details related to the neural network of the GRU model 112F are skipped for the sake of brevity of the disclosure.

The hybrid diluted convolution encoder 112G may be applied on multiple time-frequency spectrums for extraction of one or more statistical features associated with each of the determined multiple time-frequency spectrums. In an embodiment, the hybrid diluted convolution encoder 112G may be a neural network. Details related to the neural network associated with the hybrid diluted convolution encoder 112G are similar to the details of the neural network of the AI model 112A. Hence, the details related to the neural network of the hybrid diluted convolution encoder 112G are skipped for the sake of brevity of the disclosure.

The generator model 112H may be applied on selected inhale-exhale pause sample and one or more statistical features associated with each of the determined multiple time-frequency spectrums for generation of a set of signals. In an embodiment, the generator model 112H may be a neural network. Details related to the neural network associated with the generator model 112H are similar to the details of the neural network of the AI model 112A. Hence, the details related to the neural network of the generator model 112H are skipped for the sake of brevity of the disclosure.

The GGAE model 1121 may be applied on the generated flow volume curve for determination of a breathing condition. In an embodiment, the GGAE model 1121 may be a neural network. Details related to the neural network associated with the GGAE model 1121 are similar to the details of the neural network of the AI model 112A. Hence, the details related to the neural network of the GGAE model 1121 are skipped for the sake of brevity of the disclosure.

The SVD model 112J may be applied on the generated flow volume curve and the determined breathing condition for determination of a chronic disease condition. In an embodiment, the SVD model 112J may be a neural network. Details related to the neural network associated with the SVD model 112J are similar to the details of the neural network of the AI model 112A. Hence, the details related to the neural network of the SVD model 112J are skipped for the sake of brevity of the disclosure.

The transformer encoder 112K may be applied on a set of correlated features for determination of a vocal disorder. In an embodiment, the transformer encoder 112K may be a neural network. Details related to the neural network associated with the transformer encoder 112K are similar to the details of the neural network of the AI model 112A. Hence, the details related to the neural network of the transformer encoder 112K are skipped for the sake of brevity of the disclosure.

The discriminator model 112L may be applied on a generated first signal. Based on the application of the discriminator model 112L, an optimized signal may be generated. The discriminator model 112L may be a neural network. Details related to the neural network associated with the discriminator model 112L are similar to the details of the neural network of the AI model 112A. Hence, the details related to the neural network of the discriminator model 112L are skipped for the sake of brevity of the disclosure.

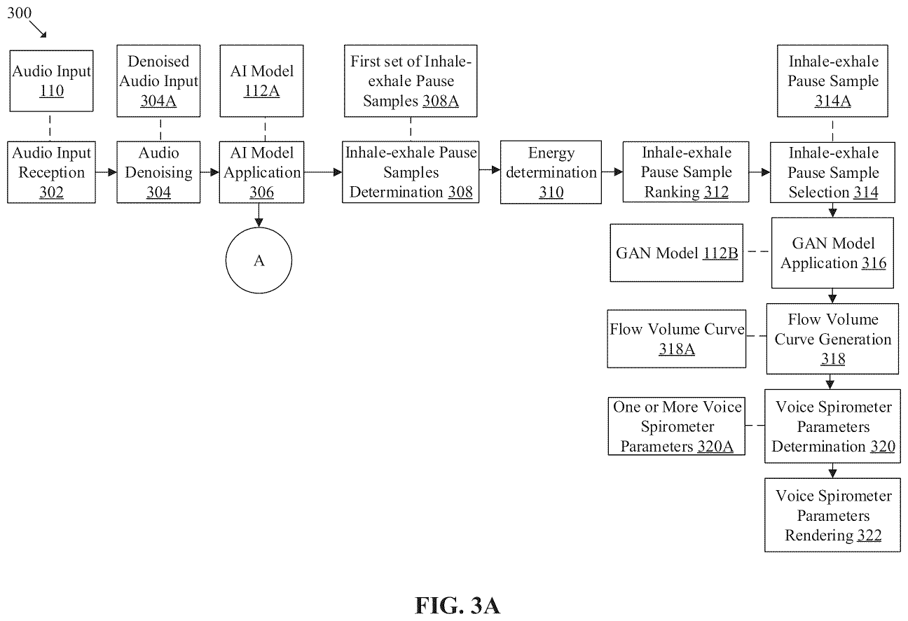

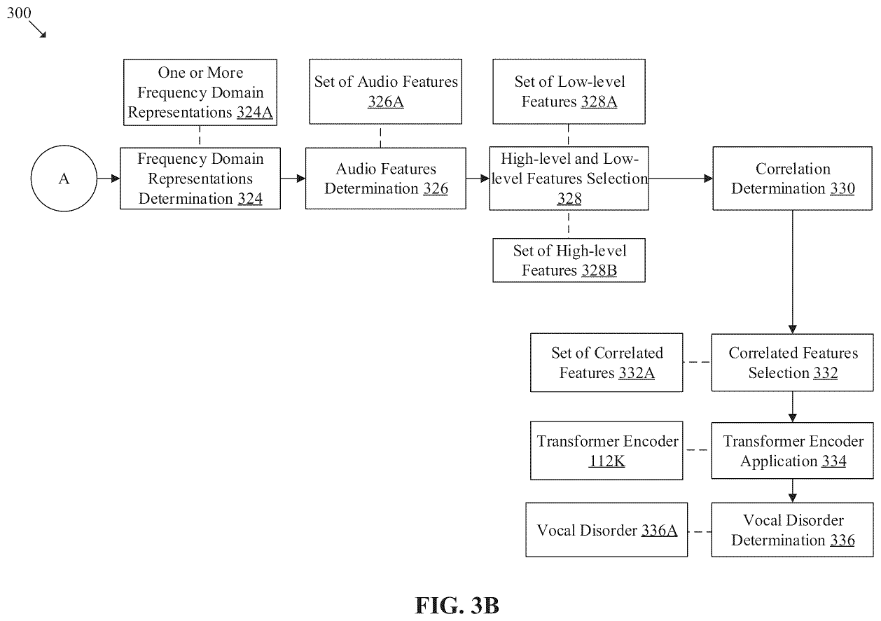

In operation, the electronic device 102 may be configured to receive the audio input associated with the user 114. For example, the electronic device 102 may include an application installed on the electronic device 102 to receive the audio input associated with the user 114. An instruction to determine the one or more voice spirometer parameters for the user 114 may also be received along with the audio input 110 received by the electronic device 102. Details related to reception of the audio input are further provided, for example, in FIG. 3A (at 302).

The electronic device 102 may be configured to apply the Artificial Intelligence (AI) model 112A on the received audio input 110. The received audio input 110 may be provided as an input to the AI model 112A. Details related to the application of AI model 112A are further provided for example, in FIG. 3A (at 306).

The electronic device 102 may be configured to determine the first set of inhale-exhale pause samples based on the application of the AI model 112A, wherein each inhale-exhale pause sample of the determined first set of inhale-exhale pause samples may correspond to the time interval between consecutive inhale and exhale breathlessness samples. In an embodiment, a threshold frequency associated with the audio input 110 may be predicted based on the application of the AI model 112A. The predicted threshold frequency may be used to segment the audio input 110 into the first set of inhale-exhale pause samples. Details related to determination of the first set of inhale-exhale pause samples are further provided, for example, in FIG. 3A (at 308).

The electronic device 102 may be configured to select the inhale-exhale pause sample from the first set of inhale-exhale pause samples. The selected inhale-exhale pause sample may be an unhealthy inhale-exhale pause sample. In an example, an inhale-exhale pause sample of longest duration may be selected from the first set of inhale-exhale pause samples. In an embodiment, energy based ranking may be used for selection of the inhale-exhale pause sample. Details related to selection of the inhale-exhale pause sample are further provided, for example, in FIG. 3A (at 314).

The electronic device 102 may be configured to apply the GAN model 112B on the selected inhale-exhale pause sample. Herein, the selected inhale-exhale pause sample may be provided as an input to the GAN model 112B. Details related to application of the GAN model 112B are further provided, for example, in FIG. 3A (at 316).

The electronic device 102 may be configured to generate the flow volume curve associated with the selected inhale-exhale pause sample based on the application of the GAN model 112B. The flow volume curve 318A may depict a variation of an inspiratory and an expiratory flow with time. Details related to the generation of the flow volume curve are further provided, for example, in FIG. 3A (at 318).

The electronic device 102 may be configured to determine one or more voice spirometer parameters based on the generated flow volume curve. The generated flow volume curve may be analyzed to determine the one or more voice spirometer parameters. In an embodiment, the determined one or more voice spirometer parameters may include at least one of a forced expiratory flow (FEF), a forced expiratory volume (FEV), a forced vital capacity (FVC), a pulmonary function value (PFV), a total lung capacity (TLC), a ratio of FEV to FVC, or breathlessness data. Details related to the determination of the one or more voice spirometer parameters are further provided, for example, in FIG. 3A (at 320).

The electronic device 102 may be configured to render the determined one or more voice spirometer parameters on a display device associated with the electronic device. In an example, the electronic device 102 may be associated with a healthcare professional. The healthcare profession may view the rendered one or more voice spirometer parameters to decide a course of treatment to the user 114. In some embodiments, the generated flow volume curve and the determined one or more voice spirometer parameters may be used to determine a breathing condition and a chronic disease condition. Further, in some embodiments, the electronic device 102 may determine a vocal disorder. In an example, the determined one or more voice spirometer parameters may be FVC as “3.17”, FEV as “2.47”, FEF as “3.37”, TLC as “5.17” ratio of FEV to FVC in percentage as “65”, PEF as “7.5”. Further, the electronic device 102 may determine a breathlessness intensity as “5” and a vocal cord disorder as “grade 1”. The determined one or more voice spirometer parameters, the breathlessness intensity, and the vocal cord disorder may be rendered on the display device. Details related to the rendering of the one or more voice spirometer parameters are further provided, for example, in FIG. 3A (at 322).

The disclosed electronic device 102 may be used for chronic pulmonary disease prediction from audio input based on inhale-exhale pause samples using artificial intelligence. The electronic device 102 of the present disclosure may determine the first set of inhale-exhale pause samples based on the application of the AI model 112A to the received audio input 110. Thereafter, the electronic device 102 may select the inhale-exhale pause sample from the first set of inhale-exhale pause samples. The application of the AI model 112A may reduce a processing time needed for selection of the inhale-exhale pause sample. The selected inhale-exhale pause sample may be applied to the GAN model 112B to generate the flow volume curve. The flow volume curve may be used to determine the one or more voice spirometer parameters.

In some embodiments, the generated flow volume curve and the determined one or more voice spirometer parameters may be used to determine a breathing condition and a chronic disease condition. Further, in some embodiments, the electronic device 102 may determine a vocal disorder. Therefore, the disclosed electronic device 120 may be used effortlessly by patients as well as doctors or nurses for determination of the one or more voice spirometer parameters. Also, as the AI model 112A may automatically select the inhale-exhale pause sample, time needed in manual selection of the inhale-exhale pause sample may be reduced. Moreover, the disclosed electronic device 102 may generate an accurate flow volume curve that may be used for accurate interpretation of the one or more voice spirometer parameters. Furthermore, the patients may use the disclosed electronic device 102 for self-diagnosis of the chronic disease condition. Thus, the disclosed electronic device 102 may need minimal medical expert intervention during the diagnosis of the chronic disease condition or the vocal disorder. That is, the disclosed electronic device 102 may enable an early diagnosis of the chronic disease condition in a time efficient and cost efficient manner.

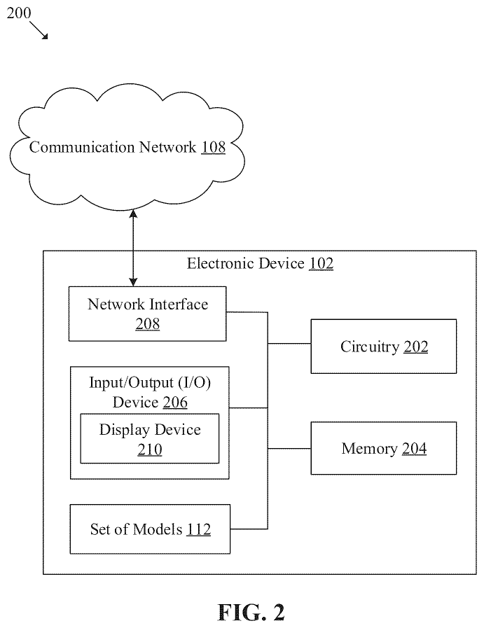

FIG. 2 is a block diagram that illustrates an exemplary electronic device of FIG. 1, in accordance with an embodiment of the disclosure. FIG. 2 is explained in conjunction with elements from FIG. 1. With reference to FIG. 2, there is shown the electronic device 102. The electronic device 102 may include circuitry 202, a memory 204, an input/output (I/O) device 206, a network interface 208, and the set of models 112. The input/output (I/O) device 206 may include a display device 210.

The circuitry 202 may include suitable logic, circuitry, and/or interfaces that may be configured to execute program instructions associated with different operations to be executed by the electronic device 102. The operations may include, for example, audio input reception, audio denoising, AI model application, inhale-exhale pause samples determination, energy determination, inhale-exhale pause sample selection, GAN model application, flow volume curve generation, and voice spirometer parameters determination. The circuitry 202 may include one or more processing units, which may be implemented as a separate processor. In an embodiment, the one or more processing units may be implemented as an integrated processor or a cluster of processors that perform the functions of the one or more specialized processing units, collectively. The circuitry 202 may be implemented based on a number of processor technologies known in the art. Examples of implementations of the circuitry 202 may be an X86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other control circuits.

The memory 204 may include suitable logic, circuitry, interfaces, and/or code that may be configured to store one or more instructions to be executed by the circuitry 202. The memory 204 may be configured to store the determined one or more voice spirometer parameters. Further, the memory 204 may be configured to store set of the audio input 110, the first set of inhale-exhale pause samples, and the generated flow volume curve. Examples of implementation of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 206 may include suitable logic, circuitry, interfaces, and/or code that may be configured to receive an input and provide an output based on the received input. For example, the I/O device 206 may receive a first user input indicative of the audio input 110. The I/O device 206 may be further configured to display the determined one or more voice spirometer parameters. The I/O device 206 may include the display device 210. Examples of the I/O device 206 may include, but are not limited to, a touch screen, a keyboard, a mouse, a joystick, a microphone, or a speaker.

The network interface 208 may include suitable logic, circuitry, interfaces, and/or code that may be configured to facilitate communication between the electronic device 102 and the server 104, via the communication network 108. The network interface 208 may be implemented by use of various known technologies to support wired or wireless communication of the electronic device 102 with the communication network. The network interface 208 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 208 may be configured to communicate via wireless communication with networks, such as the Internet, an Intranet, a wireless network, a cellular telephone network, a wireless local area network (LAN), or a metropolitan area network (MAN). The wireless communication may be configured to use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), 5th Generation (5G) New Radio (NR), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VOIP), light fidelity (Li-Fi), Worldwide Interoperability for Microwave Access (Wi-MAX), a protocol for email, instant messaging, and a Short Message Service (SMS).

The display device 210 may include suitable logic, circuitry, and interfaces that may be configured to display the determined one or more voice spirometer parameters. The display device 210 may be a touch screen which may enable a user (e.g., the user 114) to provide a user-input via the display device 210. The touch screen may be at least one of a resistive touch screen, a capacitive touch screen, or a thermal touch screen. The display device 210 may be realized through several known technologies such as, but not limited to, at least one of a Liquid Crystal Display (LCD) display, a Light Emitting Diode (LED) display, a plasma display, or an Organic LED (OLED) display technology, or other display devices. In accordance with an embodiment, the display device 210 may refer to a display screen of a head mounted device (HMD), a smart-glass device, a see-through display, a projection-based display, an electro-chromic display, or a transparent display. Various operations of the circuitry 202 for chronic pulmonary disease prediction from audio input based on inhale-exhale pause samples using artificial intelligence are described further, for example, in FIG. 3A.

FIGS. 3A and 3B diagrams that collectively illustrate an exemplary processing pipeline for chronic pulmonary disease prediction from audio input based on inhale-exhale pause samples using artificial intelligence, in accordance with an embodiment of the disclosure. FIGS. 3A and 3B are explained in conjunction with elements from FIG. 1 and FIG. 2. With reference to FIGS. 3A and 3B, there is shown an exemplary processing pipeline 300 that illustrates exemplary operations from 302 to 336. The exemplary operations 302 to 336 may be executed by any computing system, for example, by the electronic device 102 of FIG. 1 or by the circuitry 202 of FIG. 2. FIGS. 3A and 3B further include the audio input 110, a denoised audio input 304A, the AI model 112A, a first set of inhale-exhale pause samples 308A, an inhale-exhale pause sample 314A, the GAN model 112B, a flow volume curve 318A, one or more voice spirometer parameters 320A, one or more frequency domain representations 324A, a set of audio features 326A, a set of low-level features 328A, a set of high-level features 328B, a set of correlated features 332A, the transformer encoder 112K, and a vocal disorder 336A.

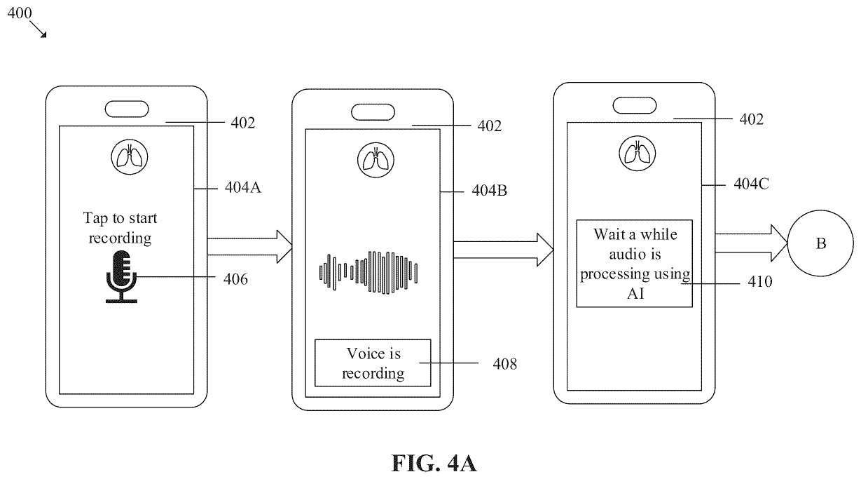

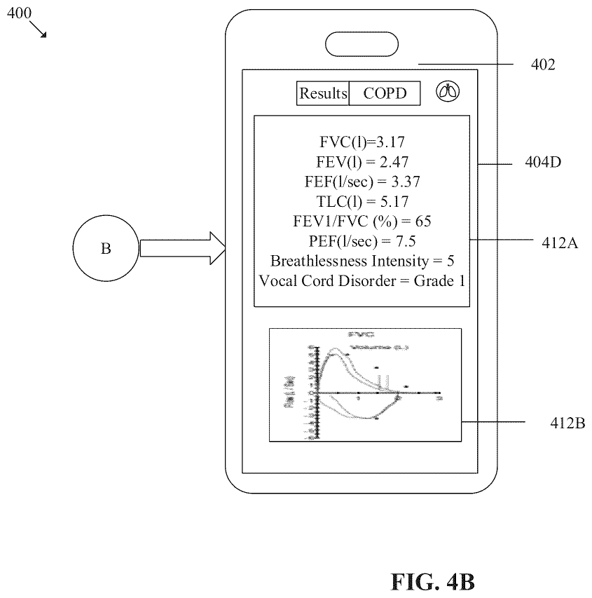

With reference to FIG. 3A, at 302, an operation of audio input reception may be executed. The circuitry 202 may receive the audio input 110 associated with the user 114. In an example, the electronic device 102 may include a microphone. The user 114 may be requested to speak in a direction of the microphone. The electronic device 102 may record live voice of the user 114 as the audio input 110. In another embodiment, the audio input 110 associated with the user 114 may be a pre-recorded voice of the user 114 that may be stored in, for example, the database 106 and/or the memory 204. The circuitry 202 may retrieve the pre-recorded voice as the audio input 110. In an embodiment, the circuitry 202 may receive a video input associated with the user 114. The circuitry 202 may further extract the audio input 110 from the received video input. Details related to the audio input reception are further provided, for example, in FIG. 4A.

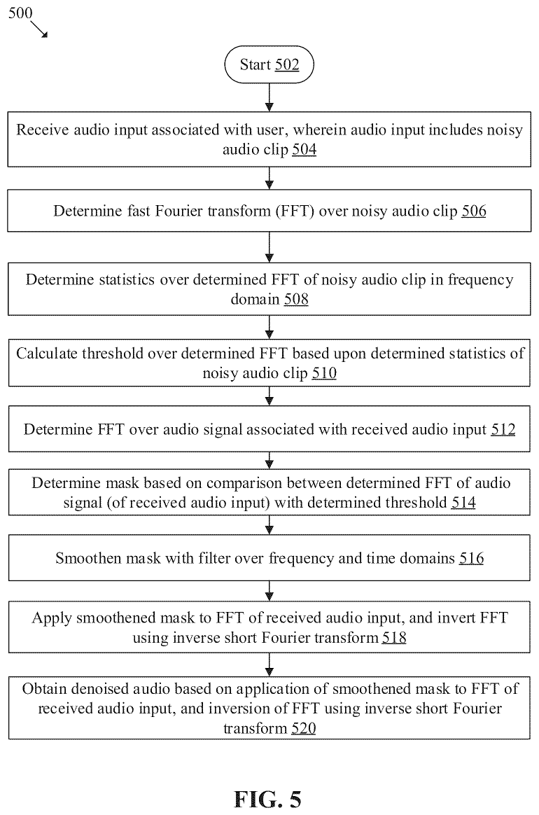

At 304, an operation for audio denoising may be executed. The circuitry 202 may be further configured to denoise the received audio input 110. It may be appreciated that the received audio input 110 may be noisy. In an example, the received audio input 110 may be an audio recording of the voice of the user 114. Herein, the received audio input 110 may include the recorded voice of the user 114 and a background noise that may be prevalent in a background of the user 114, when the voice of the user 114 was recorded. Thus, the received audio input 110 may be denoised to determine the denoised audio input 304A. Details related to the audio denoising are provided, for example, in FIG. 5.

At 306, an operation for AI model application may be executed. The circuitry 202 may be configured to apply the AI model 112A on the denoised audio input 304A. Herein, the denoised audio input 304A may be provided as an input to the AI model 112A.

In an embodiment, the circuitry 202 may be further configured to receive a set of audio samples associated with a set of users. The circuitry 202 may be further configured to extract a set of audio features associated with each audio sample of the set of audio samples. The circuitry 202 may be further configured to determine a threshold frequency associated with each audio sample of the set of audio samples. The threshold may be estimated based on adaptive thresholding estimation with statistical distribution using a frequency filtering statical artificial neural network model.

The circuitry 202 may be further configured to train the AI model 112A on the extracted set of audio features and on the determined threshold frequency associated with each audio sample of the set of audio samples. Herein, the trained AI model 112A may be applied on the received audio input 110. Further, the set of users may be users of different age groups. For example, a first subset of the set of users may be persons from “5” to “10” years of age, a second subset of the set of users may be persons from “10” to “15” years of age, a third subset of the set of users may be persons from “15” to “20” years of age, a fourth subset of the set of users may be persons from “20” to “30” years of age, a fifth subset of the set of users may be persons from “30” to “40” years of age, and a sixth subset of the set of users may be persons above of the age of “40” years. Further, a set of statistical tests such as, “T-test”, “Anova test”, “Mann-Whitney U” test, may be applied on each audio sample of the set of audio samples. Based on the application of the set of statistical tests, the set of audio features and a type of distribution may be extracted for each audio sample. Examples of the set of statistical extracted features may be, a P-value, a confidence interval, and a t-statistic. Further, the threshold frequency associated with each audio sample of the set of audio samples may be determined. Upon determination of the threshold frequency, the AI model 112A may be trained on the extracted set of audio features (for example, the set of statistical extracted features) and on the determined threshold frequency associated with each audio sample of the set of audio samples. The trained AI model 112A may be applied on the denoised audio input 304A.

In an embodiment, the AI model 112A may be a frequency-filtering statistical artificial neural network model. The frequency-filtering statistical artificial neural network model may include a set of neurons arranged in a set of layers. In an example, the AI model 112A may employ a hybrid (SGD) monarch butterfly optimization approach for optimizing of the AI model 112A.

At 308, an operation for inhale-exhale pause samples determination may be executed. The circuitry 202 may be configured to determine the first set of inhale-exhale pause samples 308A based on the application of the AI model 112A, wherein each inhale-exhale pause sample of the determined first set of inhale-exhale pause samples 308A may correspond to a time interval between consecutive inhale and exhale breathlessness samples. In an embodiment, the frequency-filtering statistical artificial neural network model may be applied on the denoised audio input 304A. Based on the application of the frequency-filtering statistical artificial neural network model, a threshold frequency associated with the audio input 110 may be predicted. The predicted threshold frequency may be used to segment the denoised audio input 304A into a set of inhale-exhale pause samples. Thereafter, the time interval of each inhale-exhale pause sample of the set of inhale-exhale pause samples may be compared with a predetermined threshold duration. The first set of inhale-exhale pause samples 308A may be determined, based on the comparison. Herein, the first set of inhale-exhale pause samples 308A may be a subset of the set of inhale-exhale pause samples, such that time duration of each of the first set of inhale-exhale pause samples 308A may be greater than the predetermined threshold duration. Details related to determination of the first set of inhale-exhale pause samples 308A are further provided, for example, in FIG. 5.

At 310, an operation for energy determination may be executed. In an embodiment, the circuitry 202 may be configured to determine an energy associated with each inhale-exhale pause sample of the first set of inhale-exhale pause samples 308A. It may be appreciated that an energy of an inhale-exhale pause sample may be determined by summing squares of amplitude of the corresponding inhale-exhale pause sample at each point. Further, the sum may be divided by a length of the corresponding inhale-exhale pause sample. Thus, the energy associated with each inhale-exhale pause sample of the first set of inhale-exhale pause samples 308A may be determined according to an equation (1):

$$E = \sqrt{\frac{( \int {s ( t )}^{2} d ( t ) )}{T}} \qquad ( 1 )$$

where “E” may be the energy, “T” may be length of the inhale-exhale pause sample, and “s(t)” may be the inhale-exhale pause sample.

At 312, an operation for inhale-exhale pause sample ranking may be executed. In an embodiment, the circuitry 202 may be configured to rank each inhale-exhale pause sample of the first set of inhale-exhale pause samples 308A based on the determined energy. It may be appreciated that higher the determined energy of an inhale-exhale pause sample, higher may be the ranking of the inhale-exhale pause sample. For example, the first set of inhale-exhale pause samples 308A may include an inhale-exhale pause samples “X”, an inhale-exhale pause samples “Y”, and an inhale-exhale pause samples “Z”. The energy of the inhale-exhale pause samples “X” may be “80” units. The energy of the inhale-exhale pause samples “Y” may be “70” units, while the energy of the inhale-exhale pause samples “Z” may be “90” units. Thus, the inhale-exhale pause samples “Z” may be ranked as “1”, the inhale-exhale pause samples “X” may be ranked as “2”, and the inhale-exhale pause samples “Y” may be ranked as “3”.

At 314, an operation for inhale-exhale pause sample selection may be executed. The circuitry 202 may be configured to select the inhale-exhale pause sample 314A from the first set of inhale-exhale pause samples 308A. In an embodiment, the inhale-exhale pause sample 314A may be selected from the first set of inhale-exhale pause samples 308A based on the ranking. For example, in case, the inhale-exhale pause sample 314A has the highest energy amongst the first set of inhale-exhale pause samples 308A, the inhale-exhale pause sample 314A may be selected. For example, the first set of inhale-exhale pause samples 308A may include an inhale-exhale pause sample “A”, an inhale-exhale pause sample “B”, an inhale-exhale pause sample “C”, and an inhale-exhale pause sample “D”. The energy of the inhale-exhale pause sample “A” may be “50” units. The energy of the inhale-exhale pause sample “B” may be “60” units. The energy of the inhale-exhale pause sample “C” may be “82” units. The energy of the inhale-exhale pause sample “D” may be “75” units. Thus, the inhale-exhale pause samples “C” may be ranked as “1”. Further, the inhale-exhale pause sample “C” may be selected.

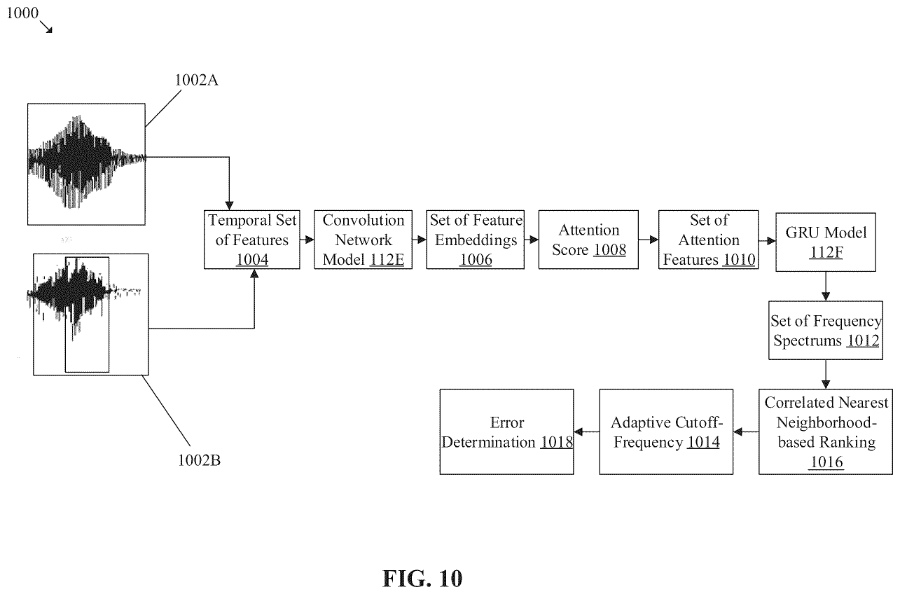

At 316, an operation for GAN model application may be executed. The circuitry 202 may be configured to apply the GAN model 112B on the selected inhale-exhale pause sample 314A. Herein, the selected inhale-exhale pause sample 314A may be provided as an input to the GAN model 112B. Details related to the application of GAN model 112B are further provided, for example, in FIG. 10.

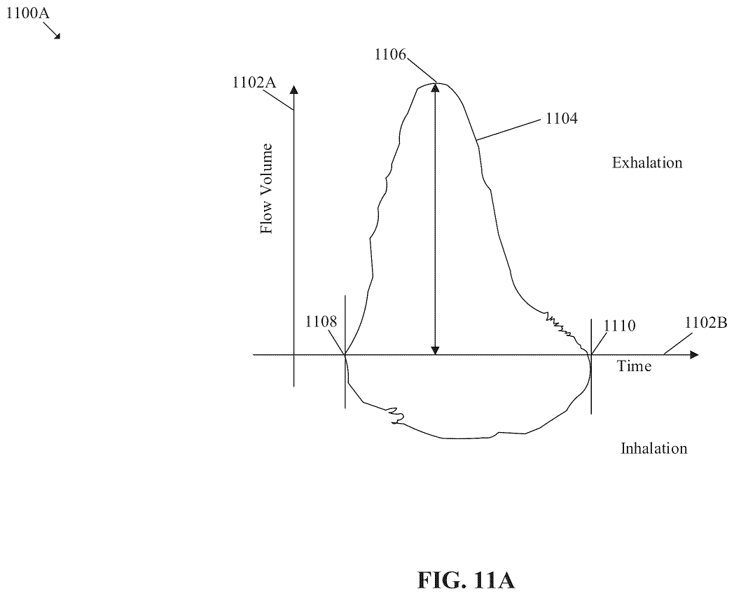

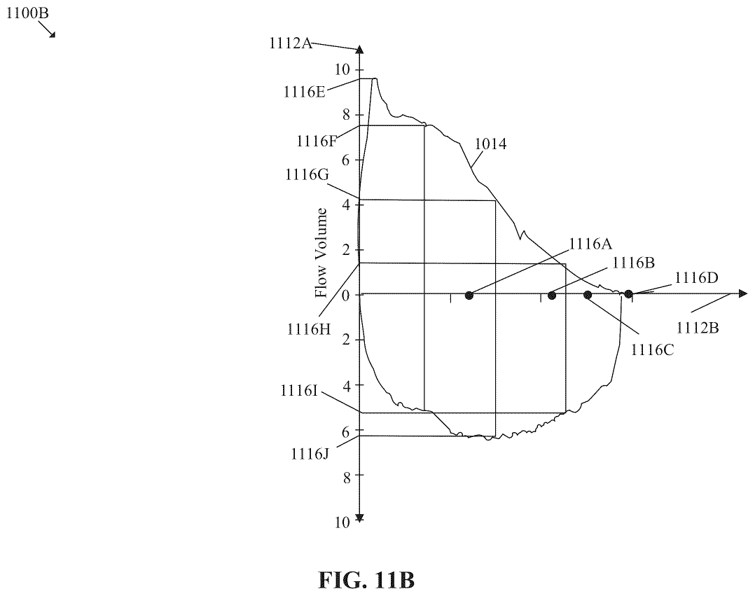

At 318, an operation for flow volume curve generation may be executed. The circuitry 202 may be configured to generate the flow volume curve 318A associated with the selected inhale-exhale pause sample 314A based on the application of the GAN model 112B. It may be appreciated that the flow volume curve 318A may be a graph of inspiratory and expiratory flow along a “Y” axis against volume along a “X” axis. Details related to the generation of the flow volume curve 318A are further provided, for example, in FIG. 11A.

At 320, an operation for voice spirometer parameters determination may be executed. The circuitry 202 may be configured to determine the one or more voice spirometer parameters 320A based on the generated flow volume curve 318A. The generated flow volume curve 318A may be analyzed to determine the one or more voice spirometer parameters 320A.

In an embodiment, the determined one or more voice spirometer parameters 320A may include at least one of a forced expiratory flow (FEF), a forced expiratory volume (FEV), a forced vital capacity (FVC), a pulmonary function value (PFV), a total lung capacity (TLC), a ratio of FEV to FVC, or breathlessness data. The FEF may be speed of air coming out of lungs during a forced expiration. To determine the FEF, the flow volume curve 318A may be first divided into a set of segments. Thereafter, a flow rate in each segment may be calculated. Thereafter, the FEF may be calculated as the average of the flow rates. The FEV may be a volume of air that the user 114 may have exhaled during first “t” seconds of the forced expiration. That is, the FEV may be calculated as the volume under the curve of the flow volume curve 318A in the first “t” seconds. The FVC may be a maximum volume of air that the user 114 may exhale during the forced expiration. The FVC may be calculated as an integral of the flow volume curve 318 over an entire forced expiratory maneuver (FEM) based on a trapezoidal rule. The PFV may be determined by identification of a highest point of the flow volume curve 318A. The TLC may be a volume of air in lungs during a maximum effort in inspiration. The TLC may be calculated by determining a slope of a linear portion of the flow volume curve 318A. The breathlessness data may indicate a breathing intensity of the user 114.

In an embodiment, the circuitry 202 may be further configured to apply the GGAE model 1121 on the generated flow volume curve 318A. The circuitry 202 may be further configured to determine a breathing condition based on the application of the GGAE model 1121. Herein, the GGAE model 1121 may be applied on the generated flow volume curve 318A to determine the breathing condition.

In an embodiment, the breathing condition may be at least one of an obstructive breathing condition, a restrictive breathing condition, a pulmonary fibrosis breathing condition, or a normal breathing condition. A patient who may be suffering from the obstructive breathing condition may suffer from shortness of breath due to the fact that the patient may be unable to exhale whole air from the lungs. It may be appreciated that the lungs of the user 114 may be elastic in nature. However, in some situations the elasticity of the lungs may decrease. Therefore, the user 114 may not be able to hold an optimum volume of air in lungs. In such cases, the user 114 may have the restrictive breathing condition. The pulmonary fibrosis breathing may be a condition where tissue around air sacs of the lungs may be damaged, thickened, and/or scarred. In such cases, the patient, such as the user 114 may face difficulty in breathing. In case the user 114 may have the normal breathing condition, then the user 114 have a respiratory rate of “12” to “18” breaths per minute.

In an embodiment, the circuitry may be further configured to divide the generated flow volume curve 318A into a set of zones. The circuitry 202 may be further configured to apply the SVD model 112J on the generated flow volume curve 318A and the determined breathing condition. The circuitry 202 may be further configured to determine a chronic disease condition based on the application of the SVD model 112J. For example, the generated flow volume curve 318A may be segmented into a first zone that may be associated with a left portion of the generated flow volume curve 318A, a second zone that may be associated with a middle portion of the generated flow volume curve 318A, and a third zone that may be associated with a right portion of the generated flow volume curve 318A. The first zone, the second zone, and the third zone, the generated flow volume curve 318A, and the determined breathing condition may be provided as inputs to the SVD model 112J. Based on the application of the SVD model 112J, the chronic disease condition may be determined.

In an embodiment, the chronic disease condition may be at least one of Chronic Obstructive Pulmonary Disease (COPD), asthma, cystic fibrosis, or pulmonary fibrosis. The COPD may be also called as emphysema or chronic bronchitis and may be a type of the obstructive breathing condition that may be caused due to clogging of the lungs of the user 114 with phlegm. The asthma may be a type of the obstructive breathing condition that may be caused due to inflammation around airways of the lungs that may cause difficulty in breathing. The pulmonary fibrosis may be a type of the restrictive breathing condition that may be caused due to damage of the tissues around air sacs of the lungs of the user 114.

At 322, an operation of rendering of voice spirometer parameters may be executed. The circuitry 202 may be configured to render the determined one or more voice spirometer parameters 320A on the display device 210 associated with the electronic device 102. In some cases, the determined breathing condition and the determined chronic disease condition may be also rendered on the display device 210. In an example, the electronic device 102 may be associated with a health care professional. The healthcare profession may then administer treatment to the user 114 based on the rendered one or more voice spirometer parameters 320A, the rendered breathing condition, and the rendered chronic disease condition.

With reference to FIG. 3B, at 324, an operation of frequency domain representation determination may be executed. The circuitry 202 may be configured to determine the one or more frequency domain representations 324A of the received audio input 110. Herein, the received audio input 110 may be represented on different time-frequency domains. In an embodiment, the one or more frequency domain representations 324A may be obtained based one or more of gammatone cepstral coefficients, short time Fourier transform, Mel frequency cepstral coefficients (MFCC), log Mel spectrogram, and/or zero crossing rate.

At 326, an operation of audio feature determination may be executed. The circuitry 202 may be configured to determine the set of audio features 326A based on the determined one or more frequency domain representations 324A. The determined one or more frequency domain representations 324A may be analyzed to determine the set of audio features 326A. In an example, the set of audio features 326A may be a pitch of the received audio input 110, a loudness of the received audio input 110, and an intensity of the received audio input 110.

At 328, an operation of low-level and high-level audio features determination may be executed. The circuitry 202 may be configured to extract the set of low-level features 328A and the set of high-level features 328B associated with the received audio input 110 based on the determined set of audio features 326A. That is, determined set of audio features 326A may be divided into the set of low-level features 328A and the set of high-level features 328B. The set of low-level features 328A may include statistical features of the received audio input 110. Examples of the set of low-level features 328A may include, but are not limited to, amplitude envelope, energy, spectral centroid, spectral flux, and zero-crossing rate. Examples of the set of high-level features 328B may include, but are not limited to, audio keys, audio chords, audio rhythms, audio melody, audio tempo, audio lyrics, audio genre, and audio mood.

At 330, an operation of correlation determination may be executed. The circuitry 202 may be configured to determine a correlation of each feature of the set of low-level features 328A and the set of high-level features 328B with other features of the set of low-level features 328A and the set of high-level features 328B. Herein, the set of low-level features 328A and the set of high-level features 328B may be provided as an input to a correlation mapping function such as, a Pearson correlation function. The correlation mapping function may correlate each feature of the set of low-level features 328A and the set of high-level features 328B with other features of the set of low-level features 328A and the set of high-level features 328B to determine the correlation of the corresponding feature.

At 332, an operation of selection of correlated features may be executed. The circuitry 202 may be configured to select the set of correlated features 332A based on the determined correlation. Upon determination of the correlation of each feature of the set of low-level features 328A and the set of high-level features 328B with other features of the set of low-level features 328A and the set of high-level features 328B, a correlation coefficient (e.g., a Pearson correlation coefficient) of the correlated features may be compared with a threshold correlation value. Features of the set of low-level features 328A and the set of high-level features 328B for which the correlation coefficient (e.g., a Pearson correlation coefficient) may be higher than the threshold correlation value may be selected as the set of correlated features 332A. Thus, the set of correlated features 332A may include highly correlated features.

At 334, an operation of transformer encoder application may be executed. The circuitry 202 may be configured to apply the transformer encoder 112K on the selected set of correlated features 332A. Herein, the selected set of correlated features 332A may be provided as an input to the transformer encoder 112K.

At 336, an operation of vocal disorder determination may be executed. The circuitry 202 may be configured to determine the vocal disorder 336A based on the application of the transformer encoder 112K. Herein, the transformer encoder 112K may analyze the selected set of correlated features 332A to determine the vocal disorder 336A. The vocal disorder 336A may indicate whether the user 114 may be suffering from COPD or dysphonia. Further, in some cases, the vocal disorder 336A may indicate a severity index for the user 114 suffering from the COPD or the dysphonia.

In an embodiment, the vocal disorder 336A may be at least one of dysphonia stage 1, dysphonia stage 2, mild COPD, moderate COPD, or severe COPD. It may be appreciated that a person suffering from dysphonia may face difficulty in speaking. Further, higher a stage of the dysphonia, greater may be the difficulty faced by the patient during speaking. Furthermore, the COPD may be the type of the obstructive breathing condition that may be caused due to clogging of the lungs of the user 114 with phlegm. In some embodiments, the determined vocal disorder 336A may be rendered on the display device 210. In an example, the healthcare profession may grasp information associated with the vocal disorder 336A based on the rendering of the vocal disorder 336. As the rendered vocal disorder 336A also indicates the severity index of the COPD or the dysphonia, the healthcare professional may be able to administer proper treatment to the patient.

The disclosed electronic device 102 may be used for chronic pulmonary disease prediction from audio input based on inhale-exhale pause samples using artificial intelligence. The electronic device 102 may generate the flow volume curve 318A that may be used to determine the one or more voice spirometer parameters 320A. In some embodiments, the generated flow volume curve 318A and the determined one or more voice spirometer parameters 320A may be used to determine the breathing condition and the chronic disease condition. Further, in some embodiments, the electronic device 102 may determine the vocal disorder 336A. Therefore, the disclosed electronic device 102 may be used effortlessly by patients as well as doctors or nurses for determination of the one or more voice spirometer parameters 320A. Also, as the AI model 112A may automatically select the inhale-exhale pause sample 314A, time needed in manual selection of the inhale-exhale pause sample 314A may be reduced. Moreover, the disclosed electronic device 102 may generate an accurate flow volume curve that may be used for accurate interpretation of the one or more voice spirometer parameters 320A. Furthermore, the patients may use the disclosed electronic device 102 for self-diagnosis of the chronic disease condition. Thus, the disclosed electronic device 102 may need minimal medical expert intervention during the diagnosis of the chronic disease condition or the vocal disorder 336A. That is, the disclosed electronic device 102 may enable an early diagnosis of the chronic disease condition in a time efficient and cost efficient manner

Thus, the disclosed electronic device 102 may be useful for the patients, as the patients may not need multiple tests for the chronic pulmonary disease prediction. Further, the electronic device 102 may be effortless and easy to use. Therefore, the patients may be encouraged to perform self-diagnosis for early detection of the COPD. Further, the disclosed electronic device 102 may be useful for the nurses, as the nurses may not need to change and clean spirometer mouthpiece every time a test is performed. Further, the application of the AI model 112A may save time needed for selection of the inhale-exhale pause sample 314A from the first set of inhale-exhale pause samples 308A. Further, the disclosed electronic device 102 may be useful for the healthcare professionals, as the healthcare professional may easily interpret the generated flow volume curve 318A and the determined one or more voice spirometer parameters 320A. Thus, a misdiagnosis of the COPD may be prevented.

FIGS. 4A and 4B are diagrams that together illustrate an exemplary scenario for chronic pulmonary disease prediction from audio input based on inhale-exhale pause samples using artificial intelligence, in accordance with an embodiment of the disclosure. FIGS. 4A and 4B are described in conjunction with elements from FIG. 1, FIG. 2, FIG. 3A, and FIG. 3B. With reference to FIGS. 4A and 4B, there is shown an exemplary scenario 400. The exemplary scenario 400 may include an electronic device 402, a first user interface (UI) 404A, a first UI element 406, a second UI 404B, a second UI element 408, a third UI 404C, a third UI element 410, a fourth UI 404D, a fourth UI element 412A, and a fifth UI element 412B. The electronic device 404 may be associated with a patient, such as, the user 114 and/or a healthcare profession, such as, a doctor. A set of operations associated the scenario 400 is described herein. Further, the electronic device 404 may be similar to the electronic device 102 of FIG. 1. Therefore, a description of the electronic device 404 is omitted for the sake of brevity.

The first user interface 404A may be displayed on a display device, (such as, the display device 210 of FIG. 2) of the electronic device 402. The first user interface 404A may provide the first UI element 406 that may be tapped (or selected) to start a recording of a speech of the user 114. Once the first UI element 406 is tapped (or selected), the display device (such as, the display device 210 of FIG. 2) may display the second UI 404B. The second UI 404B may provide the second UI element 408. The second UI element 408 may notify the user 114 that a voice of the user 114 is being recorded. The recording of the voice of the user 114 may be received as the audio input 110. Once the audio input 110 is received, the AI model 112A may be applied on the received audio input and the display device (such as, the display device 210 of FIG. 2) of the electronic device 402 may display the third UI 404C. The third UI 404C may provide the third UI element 410. The third UI element 410 may notify the user 114 that the received audio input is being processed using artificial intelligence techniques to determine the one or more voice spirometer parameters (for example, the one or more voice spirometer parameters 320A of FIG. 3A). Thereafter, the display device (such as, the display device 210 of FIG. 2) of the electronic device 402 may display the fourth UI 404D. The fourth UI 404D may include the fourth UI element 412A and the fifth UI element 412B. The fourth UI element 412A may notify the user 114 or the healthcare profession about the determined one or more voice spirometer parameters 320A and the determined vocal cord disorder (e.g., the vocal disorder 336A). In an example, the fourth UI element 412A may state that the FVC for the user 114 may be “3.17”, the FEV for the user 114 may be “2.47”, the FEF for the user 114 may be “3.37”, the TLC for the user 114 may be “5.17”, the ratio of FEV to FVC in percentage for the user 114 may be “65”, the PEF for the user 114 may be “7.5”, a breathlessness intensity for the user 114 may be “5”, the vocal cord disorder for the user 114 may be “grade 1”. Further, the fifth UI element 412B may provide information associated with the generated flow volume curve (for example, the generated flow volume curve 318A of FIG. 3A). The healthcare professional may view the determined one or more voice spirometer parameters, the determined vocal cord disorder, and the generated flow volume curve to decide a course of treatment for the user 114.

Thus, the one or more voice spirometer parameters and the vocal cord disorder may be determined for the user 114 in a non-invasive manner. Further, the healthcare processional may be notified of the determined one or more voice spirometer parameters, the determined vocal cord disorder, and the generated flow volume curve. The healthcare processional may analyze the determined one or more voice spirometer parameters, the determined vocal cord disorder, and the generated flow volume curve. Further, the healthcare processional may administer treatment remotely that may save time of the user 114 and the healthcare professional.

It should be noted that the scenario 400 of FIG. 4A and FIG. 4B is for exemplary purpose and should not be construed to limit the scope of the disclosure.

FIG. 5 is a flowchart that illustrates operations of an exemplary method for denoising audio input, in accordance with an embodiment of the disclosure. FIG. 5 is described in conjunction with elements from FIG. 1, FIG. 2, FIG. 3A, FIG. 3B, FIG. 4A, and FIG. 4B. With reference to FIG. 5, there is shown a flowchart 500. The flowchart 500 may include operations from 502 to 520 and may be implemented by the electronic device 102 of FIG. 1 or by the circuitry 202 of FIG. 2. The flowchart 500 may start at 502 and proceed to 504.

At 504, audio input associated with user 114 may be received, wherein the audio input 110 may include a noisy audio clip. The circuitry 202 may be configured to receive the audio input 110 associated with the user 114. The audio input 110 may be a raw speech data associated with the user 114. In an example, the audio input 110 may include a noisy audio clip associated with the user 114. Details related to the reception of the audio input are provided, for example, in FIG. 3A.

At 506, a fast Fourier transform (FFT) may be determined over the noisy audio clip. The circuitry 202 may be configured to determine the fast Fourier transform (FFT) over the noisy audio clip. In an embodiment, the noisy audio clip may be a noise component of the received audio input 110. In another embodiment, the noisy audio clip may be generated based on an additive-white Gaussian noise (AWGN) signal. It may be appreciated that FFT may be an algorithm to calculate a discrete Fourier transform (DFT) over a signal (e.g., the noisy audio clip) to convert the signal to a frequency domain. The FFT of the received audio input 110 may be determined to obtain spectral components associated with the received audio input 110.

At 508, statistics over the determined FFT of the noisy audio clip may be determined in the frequency domain. The circuitry 202 may be configured to determine statistics over the determined FFT of the noisy audio clip in the frequency domain. For example, statistics such as, a mean and a standard deviation of the noisy audio clip (e.g., the AWGN signal) may be calculated over the determined FFT of the noisy audio clip.

At 510, a threshold may be calculated over the determined FFT based upon the statistics of the noisy audio clip. The circuitry 202 may be configured to determine the threshold over the determined FFT based on the statistics of the noisy audio clip. It may be noted that a desired sensitivity of an algorithm for denoising the received audio input 110 may be also considered for the determination of the threshold.

At 512, an FFT may be determined over an audio signal associated with the received audio input 110. The circuitry 202 may be configured to determine the fast Fourier transform (FFT) over the audio signal associated with the received audio input 110. The determination of the FFT over the audio signal may convert the audio signal to its spectral components in the frequency domain.

At 514, a mask may be determined based on a comparison between the determined FFT of the audio signal (of the received audio input 110) with the determined threshold. The circuitry 202 may be configured to determine the mask based on a comparison of the determined FFT of the audio signal (associated with the received audio input 110) with the determined threshold. For example, the mean and standard deviation of the determined FFT of the audio signal may be calculated. Thereafter, the determined mean and standard deviation of the FFT of the audio signal (of the received audio input 110) may be compared with the determined mean and standard deviation of the FFT of the noisy audio clip to determine the mask.

At 516, the mask may be smoothened with a filter over frequency and time domains. The circuitry 202 may be configured to smoothen the mask with a filter over the frequency and the time domains. For example, a smoothening filter may be applied on the mask over the frequency and the time domains to smoothen the mask.

At 518, the smoothened mask may be applied to the FFT of the received audio input 110. The circuitry 202 may be configured to apply the smoothened mask to the FFT of the received audio input 110. Thereafter, the circuitry 202 may be configured to invert the FFT of the received audio input 110 using inverse short Fourier transform. It may be noted that the application of the smoothened mask to the FFT of the received audio input 110 may remove noisy components from the FFT of the received audio input 110 to obtain a denoised FFT. Further, the inversion of the FFT of the received audio input 110 may convert the denoised FFT from the frequency domain to the time domain.

At 520, a denoised audio may be obtained based on the application of smoothened mask to the FFT of the received audio input 110, and further based on the inversion using the inverse short Fourier transform. The circuitry 202 may be configured to obtain the denoised audio based on the application of the smoothened mask to the FFT of the received audio input 110, and further based on the inversion of the FFT of the received audio input 110, using the inverse short Fourier transform. Control may pass to end.

Although the flowchart 500 is illustrated as discrete operations, such as, 504, 506, 508, 610, 512, 514, 516, 518, and 520, the disclosure is not so limited. Accordingly, in certain embodiments, such discrete operations may be further divided into additional operations, combined into fewer operations, or eliminated, depending on the implementation without detracting from the essence of the disclosed embodiments.

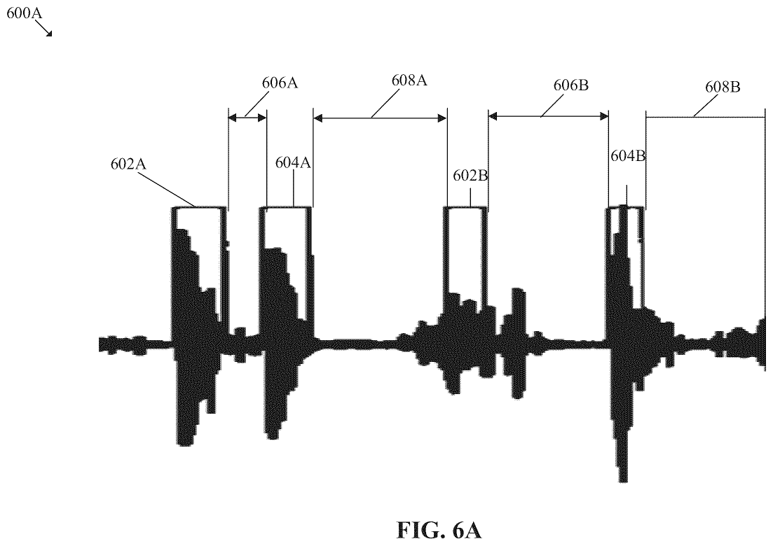

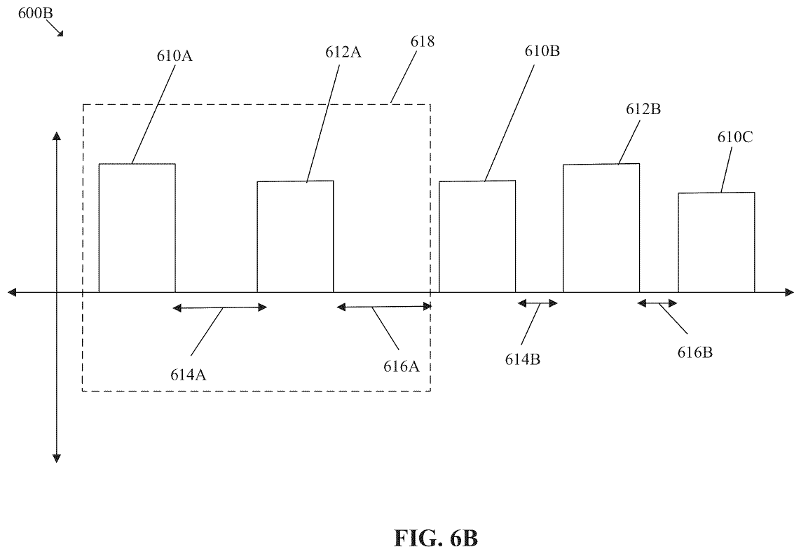

FIG. 6A is a diagram that illustrates an exemplary scenario for determination of a first set of inhale-exhale pause samples, in accordance with an embodiment of the disclosure. FIG. 6A is described in conjunction with elements from FIG. 1, FIG. 2, FIG. 3A, FIG. 3B, FIG. 4A, FIG. 4B, and FIG. 5. With reference to FIG. 6A, there is shown an exemplary scenario 600A. The exemplary scenario 600A may include an inspiratory phase 602A, an inspiratory phase 602B, an expiratory phase 604A, an expiratory phase 604B, an inspiratory pause duration 606A, an inspiratory pause duration 606B, an expiratory pause duration 608A, and an expiratory pause duration 608B. A set of operations associated the scenario 600A is described herein.

The received audio input 110 may be provided to the AI model 112A as an input. Based on the application of the AI model 112A, a set of inhale-exhale pause samples may be determined. With reference to FIG. 6A, the inspiratory phase 602A may include a set of inhale samples, the inspiratory phase 602B may include a set of inhale samples, the expiratory phase 604A may include a set of exhale samples, and an expiratory phase 604B may include a set of exhale samples. Further, the inspiratory pause duration 606A may be a pause duration between an end sample of the inspiratory phase 602A and a starting sample of the expiratory phase 604A. The inspiratory pause duration 606B may be a pause duration between an end sample of the inspiratory phase 602B and a starting sample of the expiratory phase 604B. The expiratory pause duration 608A may be a pause duration between an end sample of the expiratory phase 604A and a starting sample of the inspiratory phase 602B. Similarly, the expiratory pause duration 608B may be a pause duration between an end sample of the expiratory phase 604B and a starting sample of a next inspiratory phase.

The set of inhale-exhale pause samples may be filtered to exclude non-COPD inhale-exhale samples. In order to do so, the inspiratory pause duration 606A, the inspiratory pause duration 606B, the expiratory pause duration 608A, and the expiratory pause duration 608B may be compared with a threshold duration. Thereafter, pause duration(s), of the inspiratory pause duration 606A, the inspiratory pause duration 606B, the expiratory pause duration 608A, and the expiratory pause duration 608B, that may be lower than the threshold duration, may be eliminated from the set of inhale-exhale pause samples to determine the first set of inhale-exhale pause samples (for example, the first set of inhale-exhale pause samples 308A of FIG. 3A). Details related to determination of the first set of inhale-exhale pause samples are further provided, for example, in FIG. 6B.

It should be noted that the scenario 600A of FIG. 6A is for exemplary purpose and should not be construed to limit the scope of the disclosure.

FIG. 6B is a diagram that illustrates an exemplary scenario of a bar graph representation for the set of inhale-exhale pause samples of FIG. 6A, in accordance with an embodiment of the disclosure. FIG. 6B is described in conjunction with elements from FIG. 1, FIG. 2, FIG. 3A, FIG. 3B, FIG. 4A, FIG. 4B, FIG. 5, and FIG. 6A. With reference to FIG. 6B, there is shown an exemplary scenario 600B. The exemplary scenario 600B may include a bar graph representation for a set of inhale-exhale pause samples, detected over a time interval. The bar graph representation of the scenario 600B may include an inspiratory phase 610A, an inspiratory phase 610B, an inspiratory phase 610C, an expiratory phase 612A, an expiratory phase 612B, an inspiratory pause duration 614A, an inspiratory pause duration 614B, an expiratory pause duration 616A, an expiratory pause duration 616B, and a box 618. A set of operations associated the scenario 600B is described herein.

The received audio input 110 may be provided to the AI model 112A as an input. Based on the application of the AI model 112A, a set of inhale-exhale pause samples may be determined. With reference to FIG. 6A and FIG. 6B, the inspiratory phase 610A may correspond to the inspiratory phase 602A, the inspiratory phase 610B may correspond to the inspiratory phase 602B, the inspiratory phase 610C may correspond to a next inspiratory phase represented in FIG. 6A. The expiratory phase 612A may correspond to the expiratory phase 604A and the expiratory phase 612B may correspond to the expiratory phase 604B. Further, the inspiratory pause duration 614A may correspond to the inspiratory pause duration 606A and the inspiratory pause duration 614B may correspond to the inspiratory pause duration 608BA. The expiratory pause duration 616A may correspond to the expiratory pause duration 608A and the expiratory pause duration 616B may correspond to the expiratory pause duration 608B.

The set of inhale-exhale pause samples may be filtered to exclude non-COPD inhale-exhale samples. In order to do so, each of the inspiratory pause duration 614A, the inspiratory pause duration 614B, the expiratory pause duration 616A, and the expiratory pause duration 616B may be compared with the threshold duration. In an example, the inspiratory pause duration 614A may be higher than the threshold duration. Further, the expiratory pause duration 616A may be higher than the threshold duration. The inspiratory pause duration 614B may be lesser than the threshold duration and the expiratory pause duration 616B may also be lesser than the threshold duration. Thus, the inspiratory phase 610A and the expiratory phase 612A may be unhealthy samples. However, the inspiratory phase 610B, the inspiratory phase 610C, and the expiratory phase 612B may be healthy samples. Therefore, the inspiratory phase 610A and the expiratory phase 612A included in the box 618 may be filtered and determined as the first set of inhale-exhale pause samples (for example, the first set of inhale-exhale pause samples 308A of FIG. 3A).

It should be noted that the scenario 600B of FIG. 6B is for exemplary purpose and should not be construed to limit the scope of the disclosure.

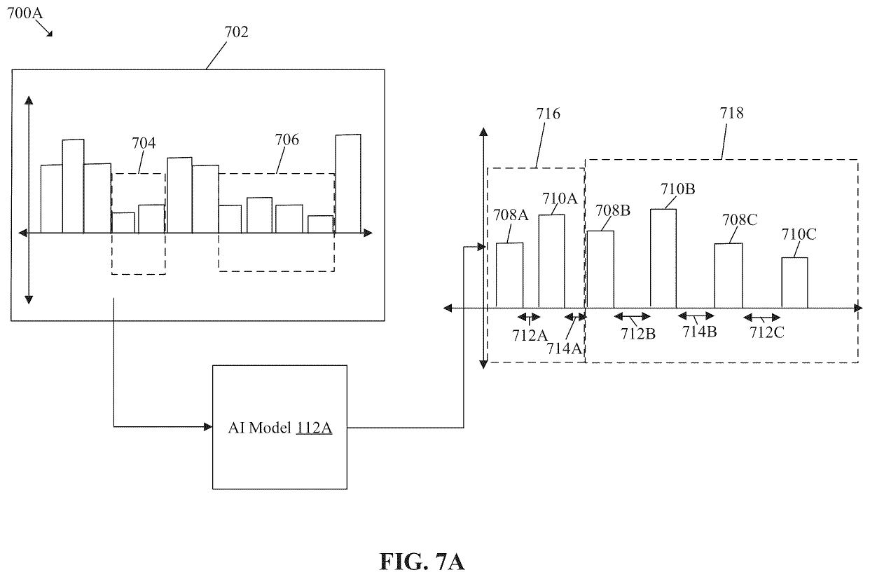

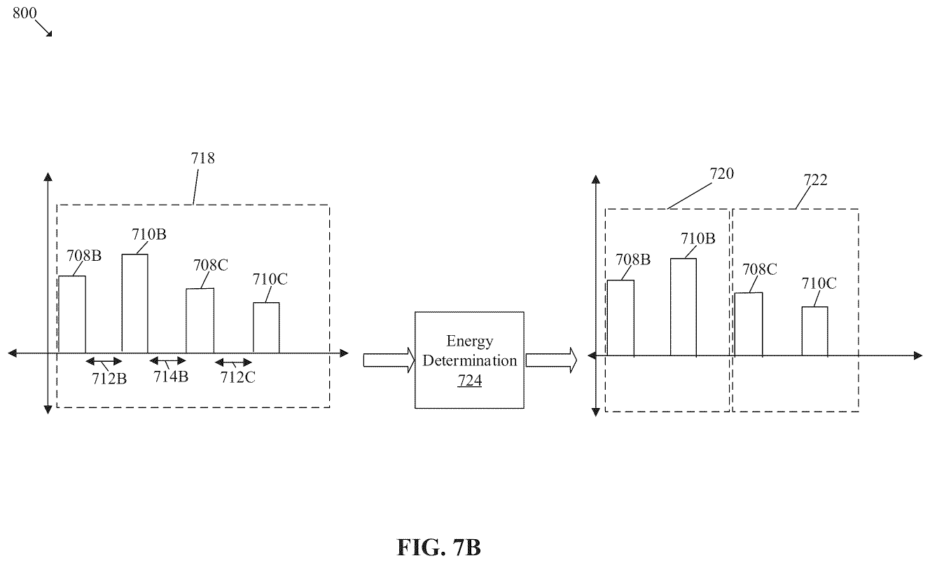

FIG. 7A is a diagram that illustrates an exemplary scenario for a determination of an intermediate set of inhale-exhale pause samples based on an application of an AI model, in accordance with an embodiment of the disclosure. FIG. 7A is described in conjunction with elements from FIG. 1, FIG. 2, FIG. 3A, FIG. 3B, FIG. 4A, FIG. 4B, FIG. 5, FIG. 6A, and FIG. 6B. With reference to FIG. 7A, there is shown an exemplary scenario 700A. The exemplary scenario 700A may include a graphical representation 702 comprising a box 704 and a box 706, the AI model 112A, an inspiratory phase 708A, an inspiratory phase 708B, an inspiratory phase 708C, an expiratory phase 710A, an expiratory phase 710B, an expiratory phase 710C, an inspiratory pause duration 712A, an inspiratory pause duration 712B, an inspiratory pause duration 712C, an expiratory pause duration 714A, an expiratory pause duration 714B, a box 716, and a box 718. A set of operations associated the scenario 700A is described herein.

The graphical representation 702 may be a bar graph of the audio input 110. Thus, the graphical representation 702 may include audio information associated with words spoken by the user 114 and inhale-exhale cycles. The box 704 may include one inhale-exhale cycle and the box 706 may include two inhale-exhale cycles. The graphical representation 702 may be provided as an input to the AI model 112A. Based on application of the AI model 112A, a set of inhale-exhale pause samples may be extracted. The set of inhale-exhale pause samples may include the inspiratory phase 708A, the inspiratory phase 708B, the inspiratory phase 708C, the expiratory phase 710A, the expiratory phase 710B, and the expiratory phase 710C. The inspiratory pause duration 712A, the inspiratory pause duration 712B, the inspiratory pause duration 712C, the expiratory pause duration 714A, and the expiratory pause duration 714B may be compared with the threshold duration. In an example, with reference to FIG. 7A, the inspiratory pause duration 712A and the expiratory pause duration 714A may be lesser than the threshold duration. Thus, the inspiratory phase 708A, the expiratory phase 710A of the box 716 may be healthy inhale-exhales samples and may be rejected. In an example, the inspiratory phase 708B, the expiratory phase 710B, the inspiratory phase 708C, and the expiratory phase 710C present in the box 718 may be unhealthy inhale-exhales samples. Thus, the inspiratory phase 708B, the expiratory phase 710B, the inspiratory phase 708C, and the expiratory phase 710C may be determined as the first set of inhale-exhale pause samples (for example, the first set of inhale-exhale pause samples 308A of FIG. 3A).

It should be noted that the scenario 700A of FIG. 7A is for exemplary purposes and should not be construed to limit the scope of the disclosure.

FIG. 7B is a diagram that illustrates an exemplary scenario for a selection of an inhale-exhale pause sample from a first set of inhale-exhale pause samples based on determination of energies, in accordance with an embodiment of the disclosure. FIG. 7B is described in conjunction with elements from FIG. 1, FIG. 2, FIG. 3A, FIG. 3B, FIG. 4A, FIG. 4B, FIG. 5, FIG. 6A, FIG. 6B, and FIG. 7A. With reference to FIG. 7B, there is shown an exemplary scenario 700B. The exemplary scenario 700B may include the inspiratory phase 708B, the inspiratory phase 708C, the expiratory phase 710B, the expiratory phase 710C, the inspiratory pause duration 712B, the inspiratory pause duration 712C, the expiratory pause duration 714B, the box 718, a box 720, and a box 722. The exemplary scenario 700B may further include an operation 724 that may be executed by any computing system, for example, by the electronic device 102 of FIG. 1 or by the circuitry 202 of FIG. 2. A set of operations associated the scenario 700B is described herein.

In an example, the inspiratory phase 708B, the expiratory phase 710B, the inspiratory phase 708C, and the expiratory phase 710C present in the box 718 may be determined as the first set of inhale-exhale pause samples (for example, the first set of inhale-exhale pause samples 308A of FIG. 3A), as described further, for example, in FIG. 7A.

At 724, an operation of energy determination may be executed. The circuitry 202 may determine an energy associated with each inhale-exhale pause sample of the first set of inhale-exhale pause samples. That is, a first energy may be determined based on an energy of the inspiratory phase 708B and the expiratory phase 710B. Further, a second energy may be determined based on an energy of the inspiratory phase 708C and the expiratory phase 710C. Thereafter, an inhale-exhale pause sample with a higher energy may be selected from the first set of inhale-exhale pause samples 308A (including, for example, a first inhale-exhale pause sample associated with the inspiratory phase 708B and the expiratory phase 710B and a second inhale-exhale pause sample associated with the inspiratory phase 708C and the expiratory phase 710C). With reference to FIG. 700B, the first energy may be greater than the second energy. Therefore, the inspiratory phase 708B and the expiratory phase 710B (together represented in the box 720) may be selected for generation of the flow volume curve (for example, the flow volume curve 318A of FIG. 3A)

It should be noted that the scenario 700B of FIG. 7B is for exemplary purpose and should not be construed to limit the scope of the disclosure.

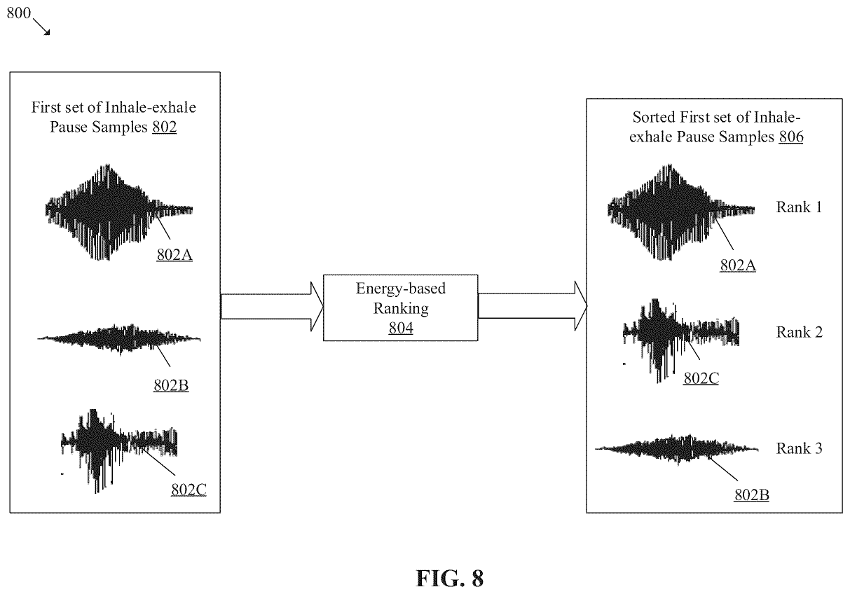

FIG. 8 is a diagram that illustrates an exemplary scenario for a selection of an inhale-exhale pause sample from a first set of inhale-exhale pause samples based on energy-based rankings, in accordance with an embodiment of the disclosure. FIG. 8 is described in conjunction with elements from FIG. 1, FIG. 2, FIG. 3A, FIG. 3B, FIG. 4A,

FIG. 4B, FIG. 5, FIG. 6A, FIG. 6B, FIG. 7A, and FIG. 7B. With reference to FIG. 8, there is shown an exemplary scenario 800. The exemplary scenario 800 may include a first set of inhale-exhale pause samples 802 and a sorted first set of inhale-exhale pause samples 806. The first set of inhale-exhale pause samples 802 may include an inhale-exhale pause sample 802A, an inhale-exhale pause sample 802B, and an inhale-exhale pause sample 802C. The exemplary scenario 800 may further include an operation 724 that may be executed by any computing system, for example, by the electronic device 102 of FIG. 1 or by the circuitry 202 of FIG. 2. A set of operations associated the scenario 800 is described herein.

At 804, an operation of energy-based ranking may be executed. The circuitry 202 may determine an energy associated with each inhale-exhale pause sample of the first set of inhale-exhale pause samples 802. That is, a first energy may be determined based on an energy of the inhale-exhale pause sample 802A. Further, a second energy may be determined based on an energy of the inhale-exhale pause sample 802B. A third energy may be determined based on an energy of the inhale-exhale pause sample 802C. Thereafter, the circuitry 202 may determine a rank of each inhale-exhale pause sample of the first set of inhale-exhale pause samples 802 based on determined energy. With reference to FIG. 8, in an example, the determined first energy may be greater than the determined second energy and the determined third energy. Further, the determined second energy may be lesser than the determined third energy. Therefore, the rank of the inhale-exhale pause sample 802A may “1”, the rank of the inhale-exhale pause sample 802B may be “3”, and the rank of the inhale-exhale pause sample 802C may be “2”. The first set of inhale-exhale pause samples 802 may be sorted based on the raking to determine the sorted first set of inhale-exhale pause samples 806. The inhale-exhale pause sample 802A having the rank as “1” may be selected from the sorted first set of inhale-exhale pause samples 806 for generation of the flow volume curve (for example, the flow volume curve 318A of FIG. 3A).

It should be noted that the scenario 800 of FIG. 8 is for exemplary purpose and should not be construed to limit the scope of the disclosure.

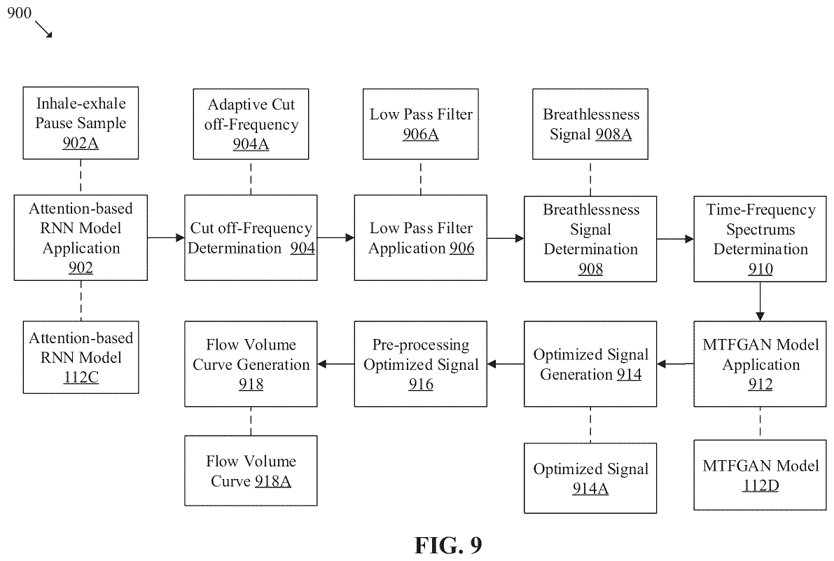

FIG. 9 is a diagram that illustrates an exemplary processing pipeline for generation of a flow volume curve, in accordance with an embodiment of the disclosure. FIG. 9 is explained in conjunction with elements from FIG. 1, FIG. 2, FIG. 3A, FIG. 3B, FIG. 4A, FIG. 4B, FIG. 5, FIG. 6A, FIG. 6B, FIG. 7A, FIG. 7B, and FIG. 8. With reference to FIG. 9, there is shown an exemplary processing pipeline 900 that illustrates exemplary operations from 902 to 918. The exemplary operations 902 to 918 may be executed by any computing system, for example, by the electronic device 102 of FIG. 1 or by the circuitry 202 of FIG. 2. FIG. 9 further includes the attention-based RNN model 112C, an inhale-exhale pause sample 902A, an adaptive cutoff-frequency 904A, a low pass filter 906A, a breathlessness signal 908A, the MTFGAN model 112D, an optimized signal 914A, and a flow volume curve 918A.

At 902, an operation of attention-based RNN model application may be executed. The circuitry 202 may apply the attention-based RNN model 112C on the inhale-exhale pause sample 902A. For example, the inhale-exhale pause sample 902A may correspond to a sample selected from the first set of inhale-exhale pause samples 802, wherein the selection may be based on an energy-based ranking of samples in the first set of inhale-exhale pause samples 802. In an example, the received audio input 110 may have a frequency of 100 Hertz with “44” samples per second. Thus, the selected inhale-exhale pause sample 902A may have a sample rate of “44,100” Hertz. The selected inhale-exhale pause sample 902A may be down-sampled to a frequency of “2450” Hertz. The down-sampled inhale-exhale pause sample 902A may be provided as an input to the attention-based RNN model 112C. Details related to the selection of the inhale-exhale pause sample 902A are further provided, for example, in FIG. 3A (at 314).

At 904, an operation of adaptive cutoff-frequency determination may be executed. The circuitry 202 may determine the adaptive cutoff-frequency 904A based on the application of the attention-based RNN model 112C. It may be noted that a cut off frequency may vary according to a set of parameters associated with the received audio input 110. The set of parameters may be a loudness, an intensity, a pitch, and the like, of the received audio input 110. Thus, for audio inputs associated with different users, the set of parameters may vary. Therefore, the adaptive cutoff-frequency 904A may be determined in accordance with the set of parameters associated with the selected inhale-exhale pause sample 902A.

At 906, an operation of low pass filter application may be executed. The circuitry 202 may apply the low pass filter 906A of the determined adaptive cutoff-frequency 904A on the selected inhale-exhale pause sample 902A based on the determined adaptive cutoff-frequency 904A. In an example, the low pass filter 906A may be a seventh-order elliptic low-pass filter of the determined adaptive cutoff-frequency 904A. Herein, the selected inhale-exhale pause sample 902A may be provided as an input to the low pass filter 906A.

At 908, an operation of breathlessness signal determination may be executed. The circuitry 202 may determine the breathlessness signal 908A based on the application of the low pass filter 906A. Upon application of the low pass filter 906A on the selected inhale-exhale pause sample 902A, the low pass filter 906A may remove unwanted signal components form the selected inhale-exhale pause sample 902A. Signal components having frequencies lesser than the determined adaptive cutoff-frequency 904A may be filtered and the unwanted signal components having frequencies higher than the determined adaptive cutoff-frequency 904A may be rejected to determine the breathlessness signal 908A.

At 910, an operation of multiple time-frequency spectrums determination may be executed. The circuitry 202 may determine multiple time-frequency spectrums based on the determined breathlessness signal 908A. Herein, one or more time-frequency spectrum methods may be applied on the determined breathlessness signal 908A to determine the multiple time-frequency spectrums. In an embodiment, the multiple time-frequency spectrums may be determined based on at least one of adaptive time-frequency transform, time-frequency distribution (TFD)-based quantification, Short time Fourier transform (STFT), pseudo-Wigner distribution, and discrete or continuous wavelet transform. In an example, the STFT may be applied on the determined breathlessness signal 908A to determine the STFT of the determined breathlessness signal 908A.

At 912, an operation of MTFGAN model application may be executed. The circuitry 202 may apply the MTFGAN model 112D on the determined multiple time-frequency spectrums. Herein, the determined multiple time-frequency spectrums may be provided as an input to the MTFGAN model 112D.

At 914, an operation of optimized signal generation may be executed. The circuitry 202 may generate the optimized signal 914A based on the application of the MTFGAN model 112D. The determined breathlessness signal 908A may include multiple signals that may be used to generate the flow volume curve 918A. The determined multiple time-frequency spectrums may be associated with the multiple signals. The MTFGAN model 112D analyze the determined multiple time-frequency spectrums to select an optimal signal from the multiple signals that may be used for the generation of the flow volume curve 918A. The optimal signal may be generated as the optimized signal 914A. The optimized signal 914A may be an optimized frequency domain signal.

At 916, an operation of pre-processing of the generated optimized signal may be executed. The circuitry 202 may pre-process the generated optimized signal 914A based on normalization. The normalization may normalize a range of the generated optimized signal 914A to a predefined range. In an example, the generated optimized signal 914A may be pre-processed based on a peak normalization. The peak normalization may ensure that amplitudes of the pre-processed optimized signal 914A may be within a predefined peak range. Thus, the normalization may clean the generated optimized signal 914A.

At 918, an operation of flow volume curve generation may be executed. The circuitry 202 may generate the flow volume curve 918A further based on the pre-processing. A maximum power and an accumulated maximum power of the pre-processed optimized signal 914A may be calculated. Based on the calculated maximum power and the calculated accumulated maximum power, the flow volume curve 918A may be generated. Thus, a healthcare professional may not need to interpret each voice spirometer parameter of the one or more voice spirometer parameters. Rather, the healthcare professional may view the flow volume curve 918A to diagnose a disease and a course of treatment for the disease. Further, one or more voice spirometer parameters associated with the flow volume curve 918A may be also determined. Thereafter, based on the generated flow volume curve 918A and the determined one or more voice spirometer parameters, the circuitry 202 may determine whether a patient such as, the user 114 suffers from COPD.

FIG. 10 is a diagram that illustrate exemplary scenario for determination of an adaptive cutoff-frequency, in accordance with an embodiment of the disclosure. FIG. 10 is described in conjunction with elements from FIG. 1, FIG. 2, FIG. 3A, FIG. 3B, FIG. 4A, FIG. 4B, FIG. 5, FIG. 6A, FIG. 6B, FIG. 7A, FIG. 7B, FIG. 8, and FIG. 9. With reference to FIG. 10, there is shown an exemplary scenario 1000. The exemplary scenario 1000A may include a global view 1002A, a local view 1002B, a temporal set of features 1004, the convolution network model 112E, a set of feature embeddings 1006, an attention score 1008, a set of attention features 1010, the GRU model 112F, a set of frequency spectrums 1012, and an adaptive cutoff-frequency 1014. The exemplary scenario 1000A further includes an operation 1016 and an operation 1018 that may be executed by any computing system, for example, by the electronic device 102 of FIG. 1 or by the circuitry 202 of FIG. 2. A set of operations associated the scenario 1000 is described herein.

The circuitry 202 may be configured to determine each of a set of high-level features and set of low-level features associated with the selected inhale-exhale pause sample (for example, the selected inhale-exhale pause sample 902A of FIG. 9A). The set of high-level features may be extracted from the global view 1002A. In an example, the global view 1002A may be high-level statistics of the selected inhale-exhale pause sample 902A. Examples of the set of high-level features may be keys, rhythms, melodies, tempo, lyrics, genres, moods, and the likes. The set of low-level features may be extracted from the local view 1002B. In an example, the local view 1002B may be low-level statistics of the selected inhale-exhale pause sample 902A. Examples of the set of high-level features may be amplitudes, envelopes, energies, spectral centroids, spectral fluxes, zero-crossing rates, and the likes.

The circuitry 202 may be further configured to determine the temporal set of features 1004 associated with the selected inhale-exhale pause sample (for example, the selected inhale-exhale pause sample 902A of FIG. 9) based on the determined set of high level features and the determined set of low level features. In an example, the determined temporal set of features 1004 may be an energy, a zero crossing rate, a maximum amplitude, a minimum energy, an instantaneous amplitude, and the like, of the selected inhale-exhale pause sample 902A.

The circuitry 202 may be further configured to apply the convolution network model 112E on the determined temporal set of features 1004. Herein, the determined temporal set of features 1004 may be provided as an input to the convolution network model 112E.

The circuitry 202 may be further configured to determine the set of feature embeddings 1006 based on the application of the convolution network model 112E. It may be appreciated that the set of feature embeddings 1006 may be a compressed vector representation of the determined temporal set of features 1004. For example, the convolution network model 112E may apply a single dimensional convolution on the determined temporal set of features 1004 to determine the compressed vector representation associated with the set of feature embeddings 1006.

The circuitry 202 may be further configured to determine the attention score 1008 associated with each of the set of feature embeddings 1006. It may be appreciated that the attention score 1008 associated with each of the set of feature embeddings 1006 may be a weighted count of attention picked up for the corresponding feature embedding.

The circuitry 202 may be further configured to extract the set of attention features 1010 based on the determined attention score 1008. For example, the determined attention score 1008 of each of the set of feature embeddings 1006 may be randomly masked to determine a corresponding masked attention score (not shown in FIG. 10). Thereafter, the circuitry 202 may extract the set of attention features 1010 based on a tensor product between the determined masked attention score of each of the set of feature embeddings 1006 and the corresponding original feature embeddings of the set of feature embeddings 1006. The circuitry 202 may be further configured to apply the GRU model 112F on the extracted set of attention features 1010. Herein, the extracted set of attention features 1010 may be provided as an input to the GRU model 112F. In an embodiment, the GRU model 112F may include recurrent layers, such as, a first layer GRU model and a second layer GRU model. The first layer of the GRU model 112F and the second layer of the GRU model 112F may be trained based on a sequential feature learning. The first layer and the second layer may learn based on the extracted set of attention features 1010. Thereafter, the circuitry 202 may be further configured to determine the set of frequency spectrums 1012 based on the application of the GRU model 112F.

At 1016, an operation for correlated nearest neighborhood-based ranking may be executed. The circuitry 202 may be further configured to rank each of the set of frequency spectrums based on an application of correlated nearest neighborhood-based ranking, wherein the adaptive cutoff-frequency 1014 may be determined further based on the ranking. Herein, the determined adaptive cutoff-frequency 1014 may be an optimized adaptive cutoff-frequency that may be determined based on the ranking. That is, the determined adaptive cutoff-frequency 1014 may be a final predicted adaptive cutoff-frequency.

At 1018, an operation for error determination may be executed. The circuitry 202 may be further configured to determine a mean squared error and a root mean square error associated with the determined adaptive cutoff-frequency 1014. The circuitry 202 may be further configured to minimize the mean squared error and the root mean square error associated with the determined adaptive cutoff-frequency 1014 using an optimization function such as, Adam optimization function, Adagra optimization function, and the like.

It should be noted that the scenario 1000 of FIG. 10 is for exemplary purposes and should not be construed to limit the scope of the disclosure.

FIGS. 11A and 11B are diagrams that illustrate exemplary scenarios of flow volume curves, in accordance with an embodiment of the disclosure. FIGS. 11A and 11B are described in conjunction with elements from FIG. 1, FIG. 2, FIG. 3A, FIG. 3B, FIG. 4A, FIG. 4B, FIG. 5, FIG. 6A, FIG. 6B, FIG. 7A, FIG. 7B, FIG. 8, FIG. 9, and FIG. 10. With reference to FIG. 11A, there is shown an exemplary scenario 1100A. The exemplary scenario 1100A may include a “Y” axis 1102A and an “X” axis 1102B, a flow volume curve 1104, a peak point 1106, a point 1108, and a point 1110. With reference to FIG. 11B, there is shown an exemplary scenario 1100B. The exemplary scenario 1100B may include a “Y” axis 1112A and an “X” axis 1112B, a flow volume curve 1114, a point 1116A, a point 1116B, a point 1116C, a point 1116D, a point 1116E, a point 1116F, a point 1116G, a point 1116H, a point 11161, and a point 1116J. A set of operations associated the scenario 1100A and 1100B are described herein.

With reference to FIG. 11A, the flow volume may be taken along the “Y” axis 1102A, and the time may be taken along the “X” axis 1102B. The flow volume curve 1104 may be plotted along the “Y” axis 1102A and the “X” axis 1102B. The peak point 1106 may be a peak flow volume of the flow volume curve 1104. The point 1108 may be associated with the TLC and the point 1110 may be associated with residual volume (RV).It may be noted that the RV may be RV a volume of air that may remain in the lungs after a maximal exhalation.

With reference to FIG. 11B, the flow volume may be taken along the “Y” axis 1112A, and the time may be taken along the “X” axis 1112B. The flow volume curve 1114 may be plotted along the “Y” axis 1112A and the “X” axis 1112B. The peak point 1106 may be a peak flow volume of the flow volume curve 1104. The point 1116A may be associated with FEV1/2, the point 1116B may be associated with FEV1, the point 1116C may be associated with FEV3, the point 1116D may be associated with FVC, and the point 1116E may be associated with the peak flow volume. Further, the point 1116F may be associated with FEF25%, the point 1116G may be associated with FEF50%, the point 1116H may be associated with FEF75%, the point 11161 may be associated with FIF10% and FIF75%, and the point 1116J may be associated with FIF50%.

It should be noted that the scenario 1100A of FIG. 11A and the scenario 1100B of FIG. 11B are for exemplary purposes and should not be construed to limit the scope of the disclosure.

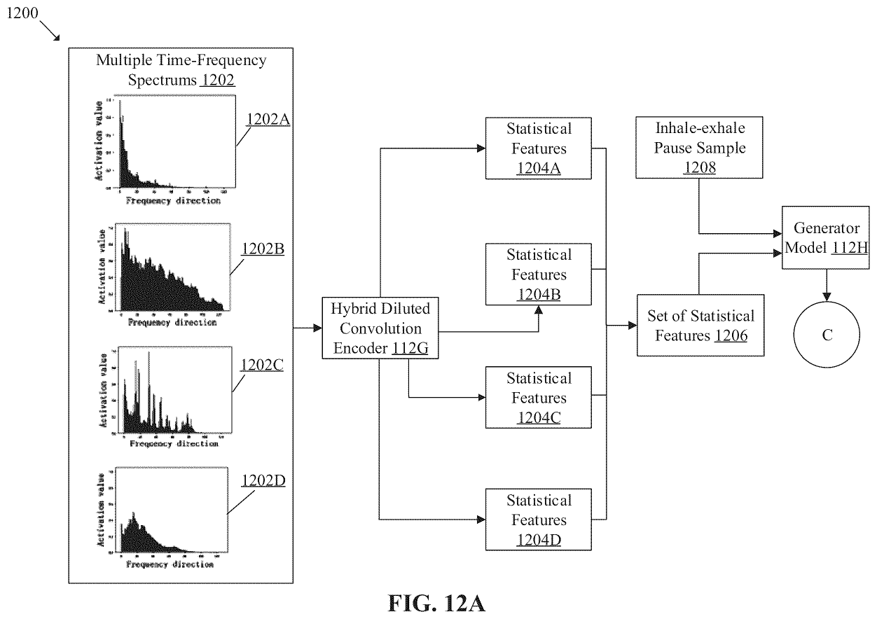

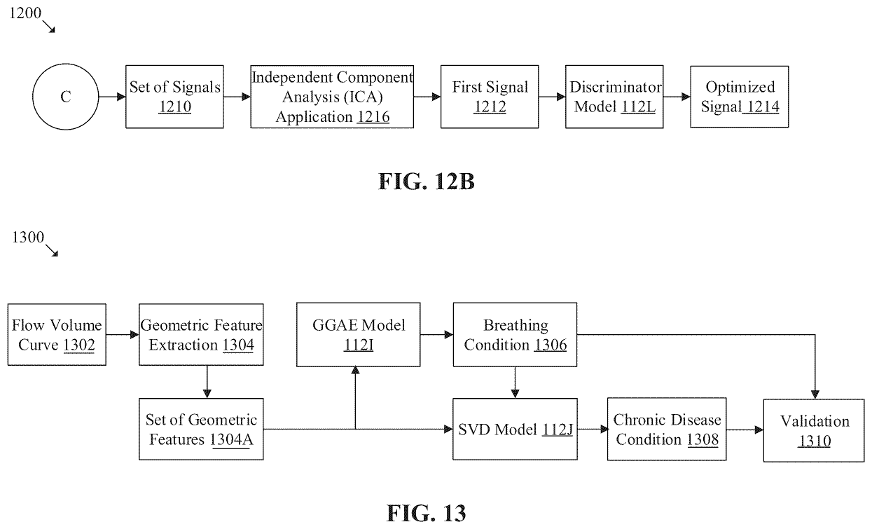

FIGS. 12A and 12B are diagrams that collectively illustrate an exemplary scenario for generation of an optimized signal, in accordance with an embodiment of the disclosure. FIGS. 12A and 12B are described in conjunction with elements from FIG. 1, FIG. 2, FIG. 3A, FIG. 3B, FIG. 4A, FIG. 4B, FIG. 5, FIG. 6A, FIG. 6B, FIG. 7A, FIG. 7B, FIG. 8, FIG. 9, FIG. 10, FIG. 11A, and FIG. 11B. With reference to FIGS. 12A and 12B, there is shown an exemplary scenario 1200. The exemplary scenario 1200 may include multiple time-frequency spectrums 1202, the hybrid diluted convolution encoder 112G, statistical features 1204A, statistical features 1204B, statistical features 1204C, statistical features 1204D, a set of statistical features 1206, an inhale-exhale pause sample 1208, the generator model 112H, a set of signals 1210, a first signal 1212, the discriminator model 112L, and an optimized signal 1214. The multiple time-frequency spectrums 1202 may include a time-frequency spectrum 1202A, a time-frequency spectrum 1202B, and a time-frequency spectrum 1202C. The exemplary scenario 1200 further includes an operation 1216 that may be executed by any computing system, for example, by the electronic device 102 of FIG. 1 or by the circuitry 202 of FIG. 2. A set of operations associated the scenario 1200 is described herein.

The circuitry 202 may apply the hybrid diluted convolution encoder 112G on the determined multiple time-frequency spectrums 1202. The determined multiple time-frequency spectrums 1202 may be provided as an input to the hybrid diluted convolution encoder 112G. Details related to the determination of the multiple time-frequency spectrums 1202 are further provided, for example, in FIG. 9 (at 910).

The circuitry 202 may extract one or more statistical features associated with each of the determined multiple time-frequency spectrums 1202 based on the application of the hybrid diluted convolution encoder 112G. For example, for the time-frequency spectrum 1202A, the statistical features 1204A may be extracted. Similarly, for the time-frequency spectrum 1202B, the statistical features 1204B may be extracted, for the time-frequency spectrum 1202C, the statistical features 1204C may be extracted, and for the time-frequency spectrum 1202D, the statistical features 1204D may be extracted. In an example, the extracted one or more statistical features associated with each of the determined multiple time-frequency spectrums 1202 may include, but are not limited to, signal distributions, means, standard deviation vectors, and the like. The statistical features 1204A, the statistical features 1204B, the statistical features 1204C, and the statistical features 1204C may be included together in the set of statistical features 1206.

The circuitry 202 may apply the generator model 112H on the inhale-exhale pause sample 1208 (i.e., a selected sample, e.g., the inhale-exhale pause sample 314A) and the extracted one or more statistical features associated with each of the determined multiple time-frequency spectrums 1202. Herein, the set of statistical features 1206 may be provided as an input to the generator model 112H.

The circuitry 202 may generate the set of signals 1210 based on the application of the generator model 112H. The determined multiple time-frequency spectrums 1202 may be associated with multiple signals. In an example, for each signal of the multiple signals, the generator model 112H may generate a signal from the set of signals 1210.

At 1216, an operation for application of independent component analysis (ICA) may be executed. The circuitry 202 may apply ICA on the generated set of signals 1210. The circuitry 202 may generate the first signal 1212 based on the application of the ICA. On application of the ICA, a dimensionality of the generated set of signals 1210 may be reduced to generate the first signal 1212. The first signal 1212 may be an optimized signal from the generated set of signals 1210.

The circuitry 202 may apply the discriminator model 1112L on the generated first signal 1112, wherein the optimized signal 1214 may be generated based further on the application of the discriminator model 1112L. Herein, the generated first signal 1112 may be provided as an input to the discriminator model 1112L. The discriminator model 1112L may determine whether the generated first signal 1112 is real or fake. The discriminator model 1112L may output the optimized signal 1214 based on the determination that the generated first signal 1112 is real.

It should be noted that the scenario 1200 of FIG. 12A and the FIG. 12B is for exemplary purposes and should not be construed to limit the scope of the disclosure.

FIG. 13 is a diagram that illustrate exemplary scenario for determination of a breathing condition and a chronic disease condition, in accordance with an embodiment of the disclosure. FIG. 13 is described in conjunction with elements from FIG. 1, FIG. 2, FIG. 3A, FIG. 3B, FIG. 4A, FIG. 4B, FIG. 5, FIG. 6A, FIG. 6B, FIG. 7A, FIG. 7B, FIG. 8, FIG. 9, FIG. 10, FIG. 11A, FIG. 11B, FIG. 12A, and FIG. 12B. With reference to FIG. 13, there is shown an exemplary scenario 1300. The exemplary scenario 1300 may include a flow volume curve 1302, a set of geometric features 1304A, the GGAE model 1121, a breathing condition 1306, the SVD model 112J, and a chronic disease condition 1308. The exemplary scenario 1300 further includes an operation 1304 and an operation 1310 that may be executed by any computing system, for example, by the electronic device 102 of FIG. 1 or by the circuitry 202 of FIG. 2. A set of operations associated the scenario 1300 is described herein.

At 1304, an operation of set of geometric features determination may be executed. The circuitry 202 may be configured to determine the set of geometric features 1304A based on the generated flow volume curve 1302. Examples of the set of geometric features 1304A may be a shape, a peak sharpness, a peak smoothness, a curve, an edge, a rapid rise, a smooth fall and the like of the generated flow volume curve 1302.

The circuitry 202 may be configured to apply the GGAE model 1121 on the generated flow volume curve 1302. Herein, initially, the determined set of geometric features 1304A may be re-shaped to determine a graph structural representation comprising a set of nodes and a set of edges. Thereafter, the graph structural representation may be provided as an input to a multi-channel “1X1” convolution layer of the GGAE model 1121. An output of the multi-channel “1X1” convolution layer may be provided as input to one or more graph convolution layers of the GGAE model 1121 for feature extraction. An output of the one or more graph convolution layers may be fed to a global pooling of the GGAE model 1121. The global pooling may be connected to a set of dense layers of the GGAE model 1121. The set of dense layers may be connected to a multi-layer perceptron (MLP) associated with the GGAE model 1121. An output of the MLP may be the breathing condition 1306. The determined breathing condition 1306 may be the obstructive breathing condition, the restrictive breathing condition, the normal breathing condition, or the pulmonary fibrosis.

With reference to FIG. 13, the determined set of geometric features 1304A and the determined breathing condition 1306 may be provided as an input to the SVD model 112J. The chronic disease condition 1308 may be determined based on the application of the SVD model 112J. In an example, the determined breathing condition 1306 may be the obstructive breathing condition. In such a case, the determined chronic disease condition 1308 may be the COPD, the asthma, or the cystic fibrosis. In another example, the determined breathing condition 1306 may be the restrictive breathing condition. In such a case, the determined chronic disease condition 1308 may be the pulmonary cystic fibrosis.

At 1310, an operation of validation may be executed. The circuitry 202 may be configured to validate the determined chronic disease condition 1308 based on the determined breathing condition 1306. Herein, a F1 score, a recall, and a precision may be determined based on the determined breathing condition 1306 and the determined chronic disease condition 1308. The determined F1 score, the recall, and the precision may be fed back to the SVD model 112J. The chronic disease condition 1308 may be determined again based on the determined F1 score, the recall, and the precision.

Thus, the circuitry 202 may identify a type of curve for the generated flow volume curve 1302. Further, the circuitry 202 may determine the chronic disease condition 1308. Thus, the healthcare professional may not get confused during interpretation of the generated flow volume curve 1302.

It should be noted that the scenario 1300 of FIG. 13 is for exemplary purposes and should not be construed to limit the scope of the disclosure.

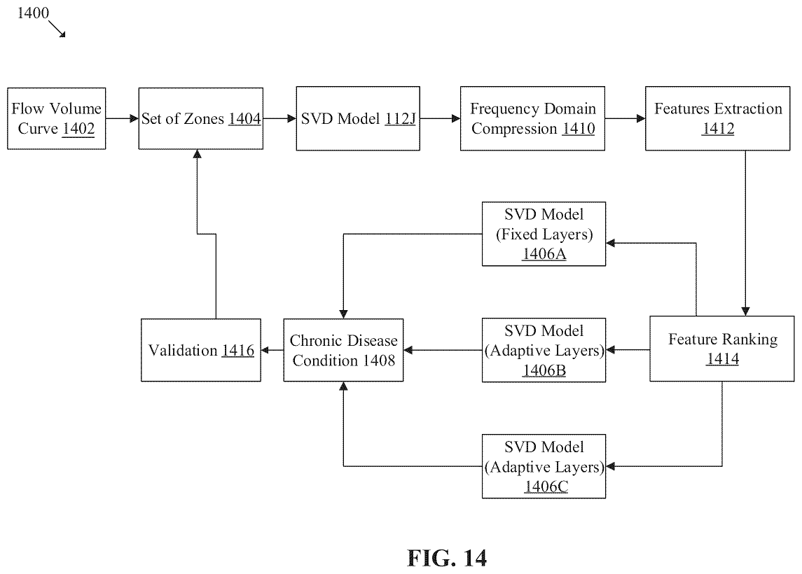

FIG. 14 is a diagram that illustrate exemplary scenario for determination of a chronic disease condition, in accordance with an embodiment of the disclosure. FIG. 14 is described in conjunction with elements from FIG. 1, FIG. 2, FIG. 3A, FIG. 3B, FIG. 4A, FIG. 4B, FIG. 5, FIG. 6A, FIG. 6B, FIG. 7A, FIG. 7B, FIG. 8, FIG. 9, FIG. 10, FIG. 11A, FIG. 11B, FIG. 12A, FIG. 12B, and FIG. 13. With reference to FIG. 14, there is shown an exemplary scenario 1400. The exemplary scenario 1400 may include a flow volume curve 1402, a set of zones 1404, the SVD model 112J, SVD model (fixed layers) 1406A, SVD model (adaptive layers) 1406B, SVD model (adaptive layers) 1406C, and a chronic disease condition 1408. The exemplary scenario 1400 further includes an operation 1410, an operation 1412, an operation 1414, and an operation 1416 that may be executed by any computing system, for example, by the electronic device 102 of FIG. 1 or by the circuitry 202 of FIG. 2. A set of operations associated the scenario 1400 is described herein.

The circuitry 202 may divide the flow volume curve 1402 into the set of zones 1404. Herein each zone may correspond to a region or a patch of the flow volume curve 1402. The SVD model 112J may be applied on the set of zones 1404 to convert an image of the flow volume curve 1402 into frequency domain.

At 1410 an operation of frequency domain compression may be executed. The circuitry 202 may apply a frequency domain compression algorithm on the converted frequency domain to determine a set of frequency domain image patches.

At 1412, an operation of features extraction may be executed. The circuitry 202 may extract a set of features based on the determined set of frequency domain image patches. In an embodiment, positional embedding and patch partitioning may be used to extract the set of features.

At 1414, an operation of feature ranking may be executed. The circuitry 202 may rank each of the extracted set of features. Based on the ranking, a top “N” number of features may be selected from the extracted set of features. The top “N” number of features may be provided as an input to the SVD model (fixed layers) 1406A, the SVD model (adaptive layers) 1406B, and the SVD model (adaptive layers) 1406C. it may be noted that a number of adaptive layers may be increased or decreased based on a length of the top “N” number of features. An output of each of the SVD model (fixed layers) 1406A, the SVD model (adaptive layers) 1406B, and the SVD model (adaptive layers) 1406C may be ensembled and a majority voting technique may be used to determine the chronic disease condition 1408.

At 1416, an operation of validation may be executed. The circuitry 202 may determine the F1 score, the recall, and the precision of the chronic disease condition 1408. Based on the determined F1 score, the recall, and the precision, a number of zones in the set of zones 1404 may be modified. For example, in case the determined F1 score, and the determined precision is lesser than a threshold F1 score and a threshold precision respectively, then the number of zones in the set of zones 1404 may be increased. Similarly, the chronic disease condition 1408 may be re-determined.

Thus, the circuitry 202 may identify a type of curve for the generated flow volume curve 1402 and determine the chronic disease condition 1408. As the generated flow volume curve 1402 may be similar for different chronic disease conditions, the identification of the generated flow volume curve 1402 and determination of the chronic disease condition 1408 may prevent the healthcare professional from misinterpretation of the generated flow volume curve 1402.

It should be noted that the scenario 1400 of FIG. 14 is for exemplary purposes and should not be construed to limit the scope of the disclosure.

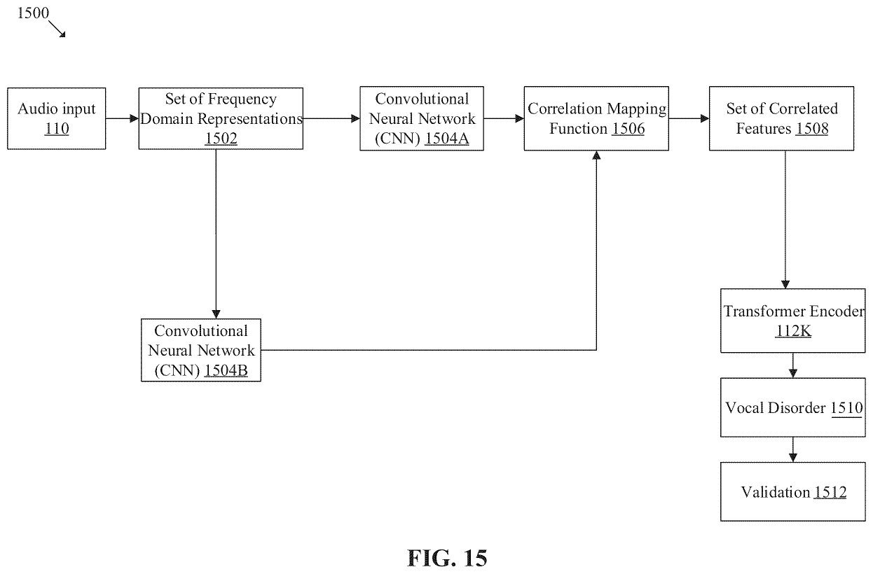

FIG. 15 is a diagram that illustrate exemplary scenario for determination of a vocal disorder, in accordance with an embodiment of the disclosure. FIG. 15 is described in conjunction with elements from FIG. 1, FIG. 2, FIG. 3A, FIG. 3B, FIG. 4A, FIG. 4B, FIG. 5, FIG. 6A, FIG. 6B, FIG. 7A, FIG. 7B, FIG. 8, FIG. 9, FIG. 10, FIG. 11A, FIG. 11B, FIG. 12A, FIG. 12B, FIG. 13, and FIG. 14. With reference to FIG. 15, there is shown an exemplary scenario 1500. The exemplary scenario 1500 may include the audio input 110, a set of frequency domain representation 1502, a convolutional neural network (CNN) 1504A, a CNN 1504B, a correlation mapping function 1506, a set of correlated features 1508, the transformer encoder 112K, and vocal disorder 1510. The exemplary scenario 1500 further includes an operation 1512 that may be executed by any computing system, for example, by the electronic device 102 of FIG. 1 or by the circuitry 202 of FIG. 2. A set of operations associated the scenario 1500 is described herein.

The circuitry 202 may determine the set of frequency domain representation 1502 of the received audio input 110. The set of frequency domain representation 1502 may be provided as an input to the CNN 1504A and the CNN 1504B. The CNN 1504A may be followed by a first bottleneck layer. An output of the CNN 1504A may be provided as an input to the first bottleneck layer. Based on the application of the first bottleneck layer, a set of low level features (for example, the set of low-level features 328A of FIG. 3B) may be extracted. The CNN 1504B may be a self-attention CNN. An output of the self-attention CNN may be provided to a second bottleneck layer. Based on the application of the second bottleneck layer a set of high level features (for example, the set of high-level features 328B of FIG. 3B) may be extracted. An output of the CNN 1504A and an output of the CNN 1504B may be provided as an input to the correlation mapping function 1506. That is, the correlation mapping function 1506 may be applied on the set of low level features (for example, the set of low-level features 328A of FIG. 3B) and the set of high level features (for example, the set of high-level features 328B of FIG. 3B). Based on the application of the correlation mapping function 1506, the set of correlated features 1508 may be determined.

The determined set of correlated features 1508 may be provided as an input to the transformer encoder 112K. Based on the application of the transformer encoder 112K, the vocal disorder 1510 may be determined. In an embodiment, the determined set of correlated features 1508 may be flattened. The flattened set of correlated features 1508 may be linearly projected to determine a linear projection. The determined linear projection may be provided as an input to the transformer encoder 112K. An output of the transformer encoder 112K may be provided to the MLP to determine the determined vocal disorder 1510.

At 1512, an operation of validation may be executed. The circuitry 202 may determine the F1 score, the recall, and the precision for the determined vocal disorder 1510. Based on the determined F1 score, the recall, and the precision, the transformer encoder 112K may be fine-tuned to update the determined vocal disorder 1510. Thus, the circuitry 202 may determine the vocal disorder 1510. The determined the vocal disorder 1510 may be at least one of the dysphonia stage 1, the dysphonia stage 2, the mild COPD, the moderate COPD, or the severe COPD. Thus, the healthcare professional may easily diagnose the dysphonia or the COPD for the patient such as, the user 114.

It should be noted that the scenario 1500 of FIG. 15 is for exemplary purposes and should not be construed to limit the scope of the disclosure.

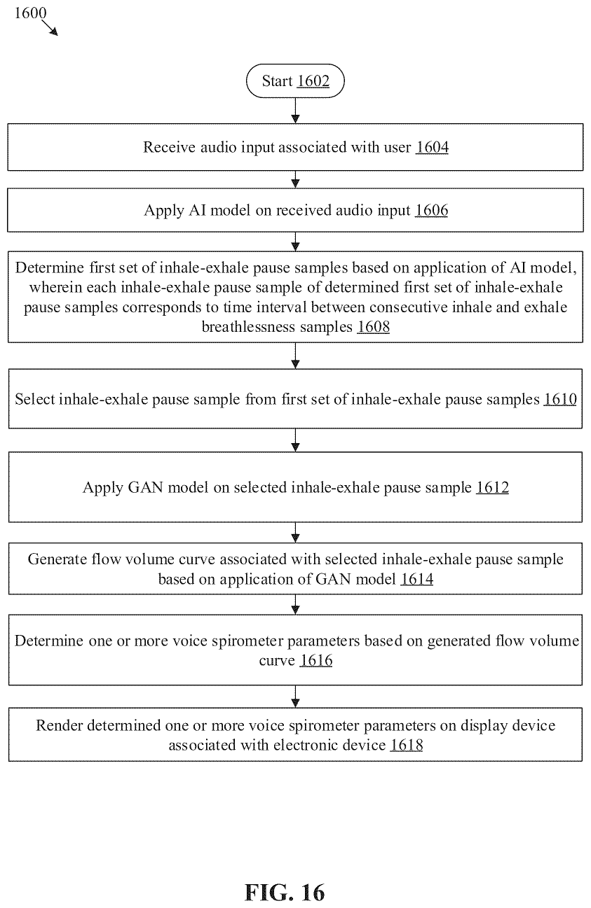

FIG. 16 is a flowchart that illustrates operations of an exemplary method for chronic pulmonary disease prediction from audio input based on inhale-exhale pause samples using artificial intelligence, in accordance with an embodiment of the disclosure. FIG. 16 is described in conjunction with elements from FIG. 1, FIG. 2, FIG. 3A, FIG. 3B, FIG. 4A, FIG. 4B, FIG. 5, FIG. 6A, FIG. 6B, FIG. 7A, FIG. 7B, FIG. 8, FIG. 9, FIG. 10, FIG. 11A, FIG. 11B, FIG. 12A, FI G. 12B, FIG. 13, FIG. 14, and FIG. 15. With reference to FIG. 16, there is shown a flowchart 1600. The flowchart 1600 may include operations from 1602 to 1618 and may be implemented by the electronic device 102 of FIG. 1 or by the circuitry 202 of FIG. 2. The flowchart 1600 may start at 1602 and proceed to 1604.

At 1604, the audio input 110 associated with the user 114 may be received. The circuitry 202 may be configured to receive the audio input 110 associated with the user 114. Details related to reception of the audio input 110 are further provided, for example, in FIG. 3A (at 302).

At 1606, the AI model 112A may be applied on the received audio input. The circuitry 202 may be configured to apply the AI model 112A on the received audio input 110. Details related to the application of AI model 112A are further provided, for example, in FIG. 3A (at 306).

At 1608, the first set of inhale-exhale pause samples 308A may be determined based on the application of the AI model 112A, wherein each inhale-exhale pause samples of the determined first set of inhale-exhale pause samples 308A may correspond to the time interval between the consecutive inhale and exhale breathlessness samples. The circuitry 202 may be configured to determine the first set of inhale-exhale pause samples 308A based on the application of the AI model 112A, wherein each inhale-exhale pause samples of the determined first set of inhale-exhale pause samples 308A may correspond to the time interval between the consecutive inhale and exhale breathlessness samples. Details related to the determination of the first set of inhale-exhale pause samples 308A are further provided, for example, in FIG. 3A (at 308).

At 1610, the inhale-exhale pause sample 314A may be selected from the first set of inhale-exhale pause samples 308A. The circuitry 202 may be configured to select the inhale-exhale pause sample 314A from the first set of inhale-exhale pause samples 308A. Details related to the selection of inhale-exhale pause sample 314A are further provided, for example, in FIG. 3A (at 314).

At 1612, the GAN model 112B may be applied on the selected inhale-exhale pause sample 314A. The circuitry 202 may be configured to apply the GAN model 112B on the selected inhale-exhale pause sample 314A. Details related to the application of the GAN model 112B are further provided, for example, in FIG. 3A (at 316).

At 1614, the flow volume curve 318A associated with the selected inhale-exhale pause sample 314A may be generated based on the application of the GAN model 112B. The circuitry 202 may be configured to generate the flow volume curve 318A associated with the selected inhale-exhale pause sample 314A based on the application of the GAN model 112B. Details related to the generation of the flow volume curve 318A are further provided, for example, in FIG. 3A (at 318).

At 1616, the one or more voice spirometer parameters 320A may be determined based on the generated flow volume curve 318A. The circuitry 202 may be configured to determine the one or more voice spirometer parameters 320A based on the generated flow volume curve 318A. Details related to the determination of the determined one or more voice spirometer parameters 320A are further provided, for example, in FIG. 3A (at 320).

At 1618, the determined one or more voice spirometer parameters 320A may be rendered on the display device 210 associated with the electronic device 102. The circuitry 202 may be configured to render the determined one or more voice spirometer parameters 320A on the display device 210 associated with the electronic device 102. Details related to the rendering of the determined one or more voice spirometer parameters 320A are further provided, for example, in FIG. 3A (at 322). Control may pass to end.

Although the flowchart 1600 is illustrated as discrete operations, such as, 1604, 1606, 1608, 1610, 1612, 1614, 1616, and 1618, the disclosure is not so limited. Accordingly, in certain embodiments, such discrete operations may be further divided into additional operations, combined into fewer operations, or eliminated, depending on the implementation without detracting from the essence of the disclosed embodiments.

Various embodiments of the disclosure may provide a non-transitory computer-readable medium and/or storage medium, and/or a computer-readable recording medium having stored thereon, computer-executable instructions executable by a machine and/or a computer to operate an electronic device (for example, the electronic device 102 of FIG. 1). Such instructions may cause the electronic device 102 to perform operations that may include receiving an audio input (for example, the audio input 110 of FIG. 1) associated with a user (for example, the user 114 of FIG. 1). The operations may further include applying an Artificial Intelligence (AI) model (for example, the AI model 112A of FIG. 1) on the received audio input. The operations may further include determining a first set of inhale-exhale pause samples (for example, the first set of inhale-exhale pause samples 308A of FIG. 3A) based on the application of the AI model 112A, wherein each inhale-exhale pause samples of the determined first set of inhale-exhale pause samples 308A may correspond to a time interval between consecutive inhale and exhale breathlessness samples. The operations may further include selecting an inhale-exhale pause sample (for example, the inhale-exhale pause sample 314A of FIG. 3A) from the first set of inhale-exhale pause samples 308A. The operations may further include applying a generative adversarial network (GAN) model (for example, the GAN model 112B of FIG. 1) on the selected inhale-exhale pause sample 314A. The operations may further include generating a flow volume curve (for example, the flow volume curve 318A of FIG. 3A) associated with the selected inhale-exhale pause sample 314A based on the application of the GAN model 112B. The operations may further include determining one or more voice spirometer parameters (for example, the one or more voice spirometer parameters 320A of FIG. 3A) based on the generated flow volume curve 318A. The operations may further include rendering the determined one or more voice spirometer parameters 320A on a display device (for example, the display device 210 of FIG. 2) associated with the electronic device 102.

Exemplary aspects of the disclosure may provide an electronic device (such as, the electronic device 102 of FIG. 1) that includes circuitry (such as, the circuitry 202). The circuitry 202 may be configured to receive an audio input (for example, an audio input 110) associated with a user (for example, the user 114 of FIG. 1). The circuitry 202 may be configured to apply an Artificial Intelligence (AI) model (for example, the AI model 112A of FIG. 1) on the received audio input 110. The circuitry 202 may be configured to determine a first set of inhale-exhale pause samples (for example, the first set of inhale-exhale pause samples 308A of FIG. 3A) based on the application of the AI model 112A, wherein each inhale-exhale pause samples of the determined first set of inhale-exhale pause samples 308A may correspond to a time interval between consecutive inhale and exhale breathlessness samples. The circuitry 202 may be configured to select an inhale-exhale pause sample (for example, the inhale-exhale pause sample 314A of FIG. 3A) from the first set of inhale-exhale pause samples 308A. The circuitry 202 may be configured to apply a generative adversarial network (GAN) model (for example, the GAN model 112B of FIG. 1) on the selected inhale-exhale pause sample 314A. The circuitry 202 may be configured to generate a flow volume curve (for example, the flow volume curve 318A of FIG. 3A) associated with the selected inhale-exhale pause sample 314A based on the application of the GAN model 112B. The circuitry 202 may be configured to determine one or more voice spirometer parameters (for example, the one or more voice spirometer parameters 320A of FIG. 3A) based on the generated flow volume curve 318A. The circuitry 202 may be configured to render the determined one or more voice spirometer parameters 320A on a display device (for example, the display device 210 of FIG. 2) associated with the electronic device 102.

In an embodiment, the circuitry 202 may be further configured to denoise the received audio input 110, wherein the application of AI model 112A may be further based on the denoised audio input.

In an embodiment, the circuitry 202 may be further configured to receive a set of audio samples associated with a set of users. The circuitry 202 may be further configured to extract a set of audio features associated with each audio sample of the set of audio samples. The circuitry 202 may be further configured to determine a threshold frequency associated with each audio sample of the set of audio samples. The circuitry 202 may be further configured train the AI model 112A on the extracted set of audio features and on the determined threshold frequency associated with each audio sample of the set of audio samples, wherein the trained AI model 112A may be applied on the received audio input 110.

In an embodiment, the AI model 112A may be a frequency-filtering statistical artificial neural network model.

In an embodiment, the circuitry 202 may be further configured to determine an energy associated with each inhale-exhale pause sample of the first set of inhale-exhale pause samples 308A. The circuitry 202 may be further configured to rank each inhale-exhale pause sample of the first set of inhale-exhale pause samples 308A based on determined energy, wherein the inhale-exhale pause sample 314A may be selected from the first set of inhale-exhale pause samples 308A based on the ranking.

In an embodiment, the circuitry 202 may be further configured to apply an attention-based recurrent neural network (RNN) model (for example, the attention-based RNN model 112C of FIG. 1) on the selected inhale-exhale pause sample 314A. The circuitry 202 may be further configured to determine an adaptive cutoff-frequency (for example, the adaptive cutoff-frequency 904A of FIG. 9) based on the application of the attention-based RNN model 112C. The circuitry 202 may be further configured to apply a low pass filter (for example, the low pass filter 906A of FIG. 9) of the determined adaptive cutoff-frequency 904A on the selected inhale-exhale pause sample 314A based on the determined adaptive cutoff-frequency 904A. The circuitry 202 may be further configured to determine a breathlessness signal (for example, the breathlessness signal 908A of FIG. 9) based on the application of the low pass filter 906A. The circuitry 202 may be further configured to determine multiple time-frequency spectrums (for example, the multiple time-frequency spectrums 1202 of FIG. 12) based on the determined breathlessness signal 908A. The circuitry 202 may be further configured to apply a multi-time frequency generative adversarial network (MTFGAN) model (for example, the MTFGAN model 112D of FIG. 1) on the determined multiple time-frequency spectrums. The circuitry 202 may be further configured to generate an optimized signal (for example, the optimized signal 914A of FIG. 9) based on the application of the MTFGAN model 112D. The circuitry 202 may be further configured to pre-process the generated optimized signal 914A based on normalization, wherein the flow volume curve 318A may be generated further based on the pre-processing.

In an embodiment, the circuitry 202 may be further configured to determine each of a set of high-level features and set of low-level features associated with the selected inhale-exhale pause sample 314A. The circuitry 202 may be further configured to determine a temporal set of features (for example, the temporal set of features 1004 of FIG. 10) associated with the selected inhale-exhale pause sample 314A based on the determined set of high level features and the determined set of low level features. The circuitry 202 may be further configured to apply a convolution network model (for example, the convolution network model 112E of FIG. 1) on the determined temporal set of features 1004. The circuitry 202 may be further configured to determine a set of feature embeddings (for example, the set of feature embeddings 1006 of FIG. 10) based on the application of the convolution network model 112E. The circuitry 202 may be further configured to determine an attention score (for example, the attention score 1008 of FIG. 10) associated with each of the set of feature embeddings 1006. The circuitry 202 may be further configured to extract a set of attention features (for example, the set of attention features 1010 of FIG. 10) based on the determined attention score 1008. The circuitry 202 may be further configured to apply a gated recurrent unit (GRU) model (for example, the GRU model 112F of FIG. 1) on the extracted set of attention features 1010. The circuitry 202 may be further configured to determine a set of frequency spectrums (for example, the set of frequency spectrums 1012 of FIG. 10) based on the application of the GRU model 112F. The circuitry 202 may be further configured to rank each of the set of frequency spectrums 1012 based on an application of correlated nearest neighborhood-based ranking, wherein the adaptive cutoff-frequency 1014 may be determined further based on the ranking.

In an embodiment, the multiple time-frequency spectrums 1202 may be determined based on at least one of adaptive time-frequency transform, TFD-Based Quantification, Short time Fourier transform (STFT), pseudo-Wigner distribution, and discrete or continuous wavelet transform.

In an embodiment, the circuitry 202 may be further configured to apply a hybrid diluted convolution encoder (for example, the hybrid diluted convolution encoder 112G) on the determined multiple time-frequency spectrums 1202. The circuitry 202 may be further configured to extract one or more statistical features (for example, the statistical features 1204A of FIG. 12A) associated with each of the determined multiple time-frequency spectrums 1202 based on the application of the hybrid diluted convolution encoder 112G. The circuitry 202 may be further configured to apply a generator model (for example, the generator model 112H of FIG. 1) on selected inhale-exhale pause sample 1208 and the extracted one or more statistical features associated with each of the determined multiple time-frequency spectrums 1202. The circuitry 202 may be further configured to generate a set of signals (for example, the set of signals 1210 of FIG. 12B) based on the application of the generator model 112H. The circuitry 202 may be further configured to apply independent component analysis (ICA) on the generated set of signals 1210. The circuitry 202 may be further configured to generate a first signal (for example, the first signal 1212 of FIG. 12B) based on the application of the ICA. The circuitry 202 may be further configured to apply a discriminator model (for example, the discriminator model 112L of FIG. 1) on the generated first signal 1212, wherein the optimized signal 1214 may be generated based further on the application of the discriminator model 112L.

In an embodiment, the determined one or more voice spirometer parameters 320A may be at least one of a forced expiratory flow (FEF), a forced expiratory volume (FEV), a forced vital capacity (FVC), a pulmonary function value (PFV), a total lung capacity (TLC), a ratio of FEV to FVC, or breathlessness data.

In an embodiment, the circuitry 202 may be further configured to apply a geometric graph autoencoder (GGAE) model (for example, the GGAE model 1121) on the generated flow volume curve 1302. The circuitry 202 may be further configured to determine a breathing condition (for example, the breathing condition 1306 of FIG. 13) based on the application of the geometric graph autoencoder model 1121.

In an embodiment, the breathing condition 1306 may be at least one of an obstructive breathing condition, a restrictive breathing condition, a pulmonary fibrosis breathing condition, or a normal breathing condition.

In an embodiment, the circuitry 202 may be further configured to divide the generated flow volume curve 1402 into a set of zones (for example, the set of zones 1404 of FIG. 14). The circuitry 202 may be further configured to apply a singular value decomposition (SVD) model (for example, the SVD model 112J of FIG. 1) on the generated flow volume curve 1402 and the determined breathing condition 1306. The circuitry 202 may be further configured to determine a chronic disease condition (for example, the chronic disease condition 1408 of FIG. 14) based on the application of the SVD model 112J.

In an embodiment, chronic disease condition 1408 may be at least one of Chronic Obstructive Pulmonary Disease (COPD), asthma, cystic fibrosis, or pulmonary fibrosis.

In an embodiment, the circuitry 202 may be further configured to determine one or more frequency domain representations (such as, the set of frequency domain representations 1502 of FIG. 15) of the received audio input 110. The circuitry 202 may be further configured to determine a set of audio features (the set of audio features 326A of FIG. 3B)) based on the determined one or more frequency domain representations 324A. The circuitry 202 may be further configured to extract a set of low-level and a set of high-level features (for example, the set of low-level features 328A and the set of high-level features 328B of FIG. 3B) associated with the received audio input 110 based on the determined set of audio features 326A. The circuitry 202 may be further configured to determine a correlation of each feature of the set of low level and a set of high level features with other features of the set of low level and a set of high level features (for example, the set of low-level features 328A and the set of high-level features 328B of FIG. 3B). The circuitry 202 may be further configured to select a set of correlated features (for example, the set of correlated features 332A of FIG. 3B) based on the determined correlation. The circuitry 202 may be further configured to apply a transformer encoder (for example, the transformer encoder 112K of FIG. 1) on the selected set of correlated features 332A. The circuitry 202 may be further configured to determine a vocal disorder (for example, the vocal disorder 336A) based on the application of the transformer encoder 112K.

In an embodiment, the one or more frequency domain representations may be obtained based one or more of: gammatone cepstral coefficients, short time Fourier transform, Mel frequency cepstral coefficients (MFCC), log Mel spectrogram, and zero crossing rate.

In an embodiment, the vocal disorder 336A may be at least one of dysphonia stage 1, dysphonia stage 2, mild COPD, moderate COPD, or severe COPD.

The present disclosure may also be positioned in a computer program product, which comprises all the features that enable the implementation of the methods described herein, and which when loaded in a computer system is able to carry out these methods. Computer program, in the present context, means any expression, in any language, code or notation, of a set of instructions intended to cause a system with information processing capability to perform a particular function either directly, or after either or both of the following: a) conversion to another language, code or notation; b) reproduction in a different material form.

While the present disclosure is described with reference to certain embodiments, it will be understood by those skilled in the art that various changes may be made, and equivalents may be substituted without departure from the scope of the present disclosure. In addition, many modifications may be made to adapt a particular situation or material to the teachings of the present disclosure without departure from its scope. Therefore, it is intended that the present disclosure is not limited to the embodiment disclosed, but that the present disclosure will include all embodiments that fall within the scope of the appended claims.

What is claimed is:

1. An electronic device, comprising:
circuitry configured to:
receive an audio input associated with a user;
apply an Artificial Intelligence (AI) model on the received audio input;
determine a first set of inhale-exhale pause samples based on the application of the AI model, wherein
each inhale-exhale pause sample of the determined first set of inhale-exhale pause samples corresponds to a time interval between consecutive inhale and exhale breathlessness samples;
select an inhale-exhale pause sample from the first set of inhale-exhale pause samples;
apply a generative adversarial network (GAN) model on the selected inhale-exhale pause sample;
generate a flow volume curve associated with the selected inhale-exhale pause sample based on the application of the GAN model;
determine one or more voice spirometer parameters based on the generated flow volume curve; and
render the determined one or more voice spirometer parameters on a display device associated with the electronic device.

2. The electronic device according to claim 1, wherein the circuitry is further configured to:
denoise the received audio input, wherein
the application of AI model is further based on the denoised audio input.

3. The electronic device according to claim 1, wherein the circuitry is further configured to:
receive a set of audio samples associated with a set of users;
extract a set of audio features associated with each audio sample of the set of audio samples;
determine a threshold frequency associated with each audio sample of the set of audio samples; and
train the AI model on the extracted set of audio features and on the determined threshold frequency associated with each audio sample of the set of audio samples, wherein
the trained AI model is applied on the received audio input.

4. The electronic device according to claim 1, wherein the AI model is a frequency-filtering statistical artificial neural network model.

5. The electronic device according to claim 1, wherein the circuitry is further configured to:
determine an energy associated with each inhale-exhale pause sample of the first set of inhale-exhale pause samples; and
rank each inhale-exhale pause sample of the first set of inhale-exhale pause samples based on determined energy, wherein
the inhale-exhale pause sample is selected from the first set of inhale-exhale pause samples based on the ranking.

6. The electronic device according to claim 1, wherein the circuitry is further configured to:
apply an attention-based recurrent neural network (RNN) model on the selected inhale-exhale pause sample;
determine an adaptive cutoff-frequency based on the application of the attention-based RNN model;
apply a low pass filter of the determined adaptive cutoff-frequency on the selected inhale-exhale pause sample based on the determined adaptive cutoff-frequency;
determine a breathlessness signal based on the application of the low pass filter;
determine multiple time-frequency spectrums based on the determined breathlessness signal;
apply a multi-time frequency generative adversarial network (MTFGAN) model on the determined multiple time-frequency spectrums;
generate an optimized signal based on the application of the MTFGAN model; and
pre-process the generated optimized signal based on normalization, wherein the flow volume curve is generated further based on the pre-processing.

7. The electronic device according to claim 6, wherein the circuitry is further configured to:
determine each of a set of high-level features and set of low-level features associated with the selected inhale-exhale pause sample;
determine a temporal set of features associated with the selected inhale-exhale pause sample based on the determined set of high level features and the determined set of low level features;
apply a convolution network model on the determined temporal set of features;
determine a set of feature embeddings based on the application of the convolution network model;
determine an attention score associated with each of the set of feature embeddings;
extract a set of attention features based on the determined attention score;
apply a gated recurrent unit (GRU) model on the extracted set of attention features;
determine a set of frequency spectrums based on the application of the GRU model; and
rank each of the set of frequency spectrums based on an application of correlated nearest neighborhood-based ranking, wherein
the adaptive cutoff-frequency is determined further based on the ranking.

8. The electronic device according to claim 6, wherein the multiple time-frequency spectrums is determined based on at least one of adaptive time-frequency transform, TFD-Based Quantification, Short time Fourier transform (STFT), pseudo-Wigner distribution, and discrete or continuous wavelet transform.

9. The electronic device according to claim 6, wherein the circuitry is further configured to:
apply a hybrid diluted convolution encoder on the determined multiple time-frequency spectrums;
extract one or more statistical features associated with each of the determined multiple time-frequency spectrums based on the application of the hybrid diluted convolution encoder;
apply a generator model on selected inhale-exhale pause sample and the extracted one or more statistical features associated with each of the determined multiple time-frequency spectrums;
generate a set of signals based on the application of the generator model;
apply independent component analysis (ICA) on the generated set of signals;
generate a first signal based on the application of the ICA; and
apply a discriminator model on the generated first signal, wherein
the optimized signal is generated based further on the application of the discriminator model.

10. The electronic device according to claim 1, wherein the determined one or more voice spirometer parameters is at least one of a forced expiratory flow (FEF), a forced expiratory volume (FEV), a forced vital capacity (FVC), a pulmonary function value (PFV), a total lung capacity (TLC), a ratio of FEV to FVC, or breathlessness data.

11. The electronic device according to claim 1, wherein the circuitry is further configured to:
apply a geometric graph autoencoder (GGAE) model on the generated flow volume curve; and
determine a breathing condition based on the application of the geometric graph autoencoder model.

12. The electronic device according to claim 11, wherein the breathing condition is at least one of an obstructive breathing condition, a restrictive breathing condition, a pulmonary fibrosis breathing condition, or a normal breathing condition.

13. The electronic device according to claim 11, wherein the circuitry is further configured to:
divide the generated flow volume curve into a set of zones;
apply a singular value decomposition (SVD) model on the generated flow volume curve and the determined breathing condition; and
determine a chronic disease condition based on the application of the SVD model.

14. The electronic device according to claim 13, wherein the chronic disease condition is at least one of Chronic Obstructive Pulmonary Disease (COPD), asthma, cystic fibrosis, or pulmonary fibrosis.

15. The electronic device according to claim 1, wherein the circuitry configured to:
determine one or more frequency domain representations of the received audio input;
determine a set of audio features based on the determined one or more frequency domain representations;
extract a set of low-level and a set of high-level features associated with the received audio input based on the determined set of audio features;
determine a correlation of each feature of the set of low level and a set of high level features with other features of the set of low level and a set of high level features;
select a set of correlated features based on the determined correlation;
apply a transformer encoder on the selected set of correlated features; and
determine a vocal disorder based on the application of the transformer encoder.

16. The electronic device according to claim 15, wherein the one or more frequency domain representations may be obtained based one or more of: gammatone cepstral coefficients, short time Fourier transform, Mel frequency cepstral coefficients (MFCC), log Mel spectrogram, and zero crossing rate.

17. The electronic device according to claim 15, wherein the vocal disorder is at least one of dysphonia stage 1, dysphonia stage 2, mild COPD, moderate COPD, or severe COPD.

18. A method, comprising:
in an electronic device:
receiving an audio input associated with a user;
applying an Artificial Intelligence (AI) model on the received audio input;
determining a first set of inhale-exhale pause samples based on the application of the AI model, wherein
each inhale-exhale pause samples of the determined first set of inhale-exhale pause samples corresponds to a time interval between consecutive inhale and exhale breathlessness samples;
selecting an inhale-exhale pause sample from the first set of inhale-exhale pause cycles;
applying a generative adversarial network (GAN) model on the selected inhale-exhale pause sample;
generating a flow volume curve associated with the selected inhale-exhale pause sample based on the application of the GAN model;
determining one or more voice spirometer parameters based on the generated flow volume curve; and
rendering the determined one or more voice spirometer parameters on a display device associated with the electronic device.

19. The method according to claim 18, further comprising:
receiving a set of audio samples associated with a set of users;
extracting a set of audio features associated with each audio sample of the set of audio samples;
determining a threshold frequency associated with each audio sample of the set of audio samples; and
training the AI model on the extracted set of audio features and the determined threshold frequency associated with each audio sample of the set of audio samples, wherein
the trained AI model is applied on the received audio input.

20. A non-transitory computer-readable medium having stored thereon, computer-executable instructions that when executed by an electronic device, causes the electronic device to execute operations, the operations comprising:
receiving an audio input associated with a user;
applying an Artificial Intelligence (AI) model on the received audio input;
determining a first set of inhale-exhale pause samples based on the application of the AI model, wherein
each inhale-exhale pause samples of the determined first set of inhale-exhale pause samples corresponds to a time interval between consecutive inhale and exhale breathlessness samples;
selecting an inhale-exhale pause sample from the first set of inhale-exhale pause cycles;
applying a generative adversarial network (GAN) model on the selected inhale-exhale pause sample;
generating a flow volume curve associated with the selected inhale-exhale pause sample based on the application of the GAN model;
determining one or more voice spirometer parameters based on the generated flow volume curve; and
rendering the determined one or more voice spirometer parameters on a display device associated with the electronic device.
